(19) United States
(12) Patent Application Publication
(10) Pub. No.: US 2022/0251517 A1
(43) Pub. Date: Aug. 11, 2022

(54) 3-D HUMAN MODEL OF COMPLEX CARDIAC ARRHYTHMIAS

(71) Applicant: University Health Network, Toronto (CA)

(72) Inventors: Sara S. Nunes de Vasconcelos, Toronto (CA); Kenneth Williams, Toronto (CA)

(21) Appl. No.: 17/668,321

(22) Filed: Feb. 9, 2022

Related U.S. Application Data
(60) Provisional application No. 63/147,744, filed on Feb. 9, 2021.

(51) Int. Cl. C12N 5/071; C12N 5/00; C12N 5/077; G01N 33/50

(52) U.S. Cl. CPC C12N 5/0697; C12N 5/0062; C12N 5/0657; C12N 5/0656; C12N 5/0068; C12N 2533/70; G01N 33/5044; C12N 2513/00; C12N 2502/1323; C12N 2502/1329; C12N 2533/56; G01N 33/5088

(57) ABSTRACT

Various embodiments are described herein for creating a 3D human heart model for modelling arrythmias, wherein the method comprises seeding a structure with a mixture of human cardiomyocytes, cardiac fibroblasts and a fibrin mixture to form cardiac tissue; applying a plating media for settlement and compaction of the cardiac tissue; and adding an arrhythmogenic media to the cardiac tissue, where the arrhythmogenic media comprises methyl-beta cyclodextrin for disrupting calcium signaling.

### CROSS-REFERENCE TO RELATED APPLICATION

This application claims the benefit of U.S. Provisional Patent Application No. 63/147,744 filed Feb. 9, 2021 and the entire contents of U.S. Provisional Patent Application No. 63/147,744 are hereby incorporated herein in its entirety.

### FIELD

Various embodiments are described herein that generally relate to application of an arrhythmogenic media to a 3D human microtissue for modelling cardiac arrhythmias.

### BACKGROUND

The following paragraphs are provided by way of background to the present disclosure. They are not, however, an admission that anything discussed therein is prior art or part of the knowledge of persons skilled in the art.

The number of patients admitted to hospitals due to arrhythmia-related medical issues has been increasing in western societies [1]. This is a result, in part, of an aging population with compounding comorbidities [1]. Currently, more than 12 million people live with potentially life-threatening arrhythmias. This is a major burden on the healthcare system where treatment of patients presenting arrhythmias accounts for more than $30 billion in direct healthcare costs [2,3]. However, there has been an absence of major advances in new anti-arrhythmic treatments, which can be partly attributed to species-specific differences in animal physiology in model organisms, such as beating rate, myofilament compositions, cardiomyocyte electrophysiology, and a poor understanding of the mechanistic basis of arrhythmia initiation, maintenance and termination [4].

Human pluripotent stem cell-derived cardiomyocytes (hiPSC-CMs) have spurred progress in studying human-relevant arrhythmias in vitro [5], including the investigation of congenital and acquired arrhythmias at the single cell level, in 2D tissues [1, 6], and more recently in 3D Engineered Heart Tissue (EHT) models [7-11]. Two-dimensional models have been useful due to their relative simplicity and ability to study some of the fundamental mechanisms underlying arrhythmias, including as re-entry circuits [1]. However, these 2D models lack the complex tissue architecture and more clinically relevant cardiac electrical activity seen in 3D models [4, 12].

Models of 3D acquired arrhythmias have shown promise as anti-arrhythmic pharmaceutical testing platforms [8-11]. However, these models: (a) implement arrhythmia induction techniques, such as tachypacing, that are associated with low success rates (˜60% arrhythmia induction), (b) lack of complexity reflecting a more tachycardic (high frequency) nature, (c) lack of effective control over the complexity of the observed arrhythmias, and (d) involve complex and lengthy preparations. Moreover, these arrhythmias self-terminate, limiting their potential in arrhythmia termination studies.

### SUMMARY OF VARIOUS EMBODIMENTS

In accordance with one aspect of the teachings herein, there is provided in at least one embodiment a method for creating a 3D human heart model for modelling arrythmias, wherein the method comprises seeding a structure with a mixture of human cardiomyocytes, cardiac fibroblasts and a fibrin mixture to form cardiac tissue; applying a plating media for settlement and compaction (i.e. self-remodeling) of the cardiac tissue; and adding an arrhythmogenic media to the cardiac tissue, where the arrhythmogenic media comprises methyl-beta cyclodextrin for disrupting calcium signaling.

In at least one embodiment, the methyl-beta cyclodextrin is conjugated to a fatty acid.

In at least one embodiment, the arrhythmogenic media is added to the cardiac tissue about seven days after the seeding is performed.

In at least one embodiment, the arrhythmogenic media is added over an increasing number of days to increase arrhythmia complexity of the cardiac tissue.

In at least one embodiment, the human cardiomyocytes comprise induced pluripotent stem cell-cardiomyocyte cells or embryonic stern cell-cardiomyocyte cells.

In at least one embodiment, the arrhythmogenic media further comprises linoleic acid, oleic acid, palmitic acid, glutamine, and/or antibiotic-antimycotic.

In at least one embodiment, the human cardiomyocytes and the fibroblasts are in a ratio of about 1:1 to about 9:1. In some cases it is preferable for the ratio to be about a 3:1 ratio.

In at least one embodiment, the seeding is performed in during a seeding time period to prevent premature fibrin gel polymerization, where the seeding time period ranges from about 5 seconds up to about 45 seconds.

In at least one embodiment, the plating media is changed every second day.

In at least one embodiment, the plating media is applied for approximately 5 to 9 days to achieve compaction. In some cases, it is preferable for the plating media to be applied for about 7 days to achieve compaction.

In at least one embodiment, the seeding comprises also using endothelial cells.

In at least one embodiment, the human cardiomyocytes include nodal cells, or cardiomyocytes of having an atrial phenotype.

In at least one embodiment, the fibrin mixture comprises a biocompatible fibrin hydrogel.

In at least one embodiment, the structure that is seeded comprises rods in at least one microwell of a heart-on-a-chip platform.

In accordance with another aspect of the teachings herein, there is provided in at least one embodiment a kit for performing tests on a human heart model, where the kit comprises: a heart-on-a-chip platform comprising at least one microwell; support elements for placement in the at least one microwell; plating media used for settlement and compaction of the cardiac tissue; and an arrhythmogenic media comprising methyl-beta cyclodextrin that is added during the formation of the cardiac tissue during use to disrupt calcium signaling.

In at least one embodiment, the kit further comprises components for seeding the at least one microwell to form cardiac tissue when these components are not otherwise available where the components include: human cardiomyocytes, cardiac fibroblasts and a fibrin mixture.

In at least one embodiment, the kit comprises a fatty acid which is used to conjugate the methyl-beta cyclodextrin.

In at least one embodiment, the components further include endothelial cells.

In at least one embodiment, the human cardiomyocyte includes nodal cells or cardiomyocytes having an atrial phenotype.

In accordance with another aspect of the teachings herein, there is provided in at least one embodiment a use of an arrhythmogenic media with a 3D human heart model for generating cardiac tissue with an arrythmia where the use comprises using methyl-beta cyclodextrin in the arrhythmogenic media.

Other features and advantages of the present application will become apparent from the following detailed description taken together with the accompanying drawings. It should be understood, however, that the detailed description and the specific examples, while indicating preferred embodiments of the application, are given by way of illustration only, since changes and modifications within the spirit and scope of the application will become apparent to those skilled in the art from this detailed description.

### BRIEF DESCRIPTION OF THE DRAWINGS

The patent or application file contains at least one drawing executed in color. Copies of this patent or patent application publication with color drawings will be provided by the Office upon request and payment of the necessary fee.

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIGS. 1A-1H generally show examples of arrhythmogenesis mediated changes in cardiac microtissue functional parameters where FIG. 1A shows a schematic of tissue generation and treatment regimen in which both hiPSC-CM (cell line 1) and human embryonic stem-cell-derived CM (cell line 2) were used; FIG. 1B shows spontaneous beat rate (Beats per minute, BPM) of tissues exposed to arrhythmogenic (Arr) or control (CTRL) media over time (N=3, n=6) assessed from brightfield microscopy videos; FIG. 10 shows representative electrophysiological phase maps of arrhythmic (bottom) and time-matched control (top) at day 5 post-treatment in which arrows (white) indicate the direction of the wavefront and the legend on the right-hand side details the color associated with specific phase (4, 0, 1, 2, 3, 4) of the calcium transient of a ventricular cardiomyocyte (scale bar, 500 μm); FIG. 1D shows representative calcium transient profile of time-matched microtissues treated with control (left) and arrhythmic (right) media for 5 days where the red scale bar represents 1 second (the white scale bar represents 500 μm); FIG. 1E shows Mean wavefronts per frame; FIG. 1F shows conduction velocity (cm/s), FIG. 1G shows cycle length (ms) of control and arrhythmic tissues over time (N=3, n=6); and FIG. 1H is a representation of assessment of the distance traveled by a wavefront in which microtissue was stimulated by a silver electrode at 3 Hz and 6V and the distance from the electrode to which point the cardiac microtissue was not electrically captured was measured(mm), the black scale bar represents 500 μm, the dotted line in the line graph indicates the average valued measured in the control tissues (N=3, n=3) and for cell line 1: significant differences between treatment days are indicated by dissimilar letters (FIGS. 1A-1H, cell line 1).

FIGS. 2A-2E generally show an example of an experimental setup used to perform measurements and experimental results showing arrhythmia induction mediated 3D differential effects in cardiac microtissues where FIG. 2A shows the electrophysiology setup that was used consisted of two SciMedia MiCams positioned above and below a petri stage that supported a petri dish that contained the tissue to allow for a 3-dimensional representation of the tissue and a perfusion circuit allowed for cell culture media to remain close to physiological 37° C. by cycling culture media through a hot water heating coil; FIG. 2B shows Electro-physiological isochrone maps of an hiPSC-CM cardiomyocyte monolayer generated from acquired Fluo-4 fluorescence data captured using the top and bottom MiCam cameras and a MatLab program; FIG. 2C shows a differential isochrone map illustrating top minus bottom activation times and a derived histogram representing the lack of differential effects observed between the isochrones maps depicted in FIG. 2B; and FIGS. 2D-2E show BrainVision Analyzer 2 snapshots of the fluorescence difference between the electrophysiological profiles captured by the top and bottom MiCam cameras of an iCell hiPSC-CM microtissue exposed to control (FIG. 2D) and arrhythmogenic (FIG. 2E) media for 5 days where the locating traces (black and red) represent the fluorescence difference of the tissue at two different points and the white scale bar represents a scale of 500 μm (FIGS. 2A-2E, cell line 1).

FIGS. 3A-3F show example experimental results that generally show that arrhythmic tissues respond to anti-arrhythmic agents and electrical defibrillation where FIG. 3A shows representative phase maps of an arrhythmic tissue prior to lidocaine treatment (left), and after 30 min incubation in lidocaine (right) where the arrows (white) indicate the direction of the wavefront and the legend on the middle of FIG. 3A details the color associated with a specific action potential phase (Scale bar, 500 μm); FIGS. 3B-3F show mean wavefronts per frame, conduction velocity (cm/s), and cycle length of arrhythmic tissues prior to and following treatment with anti-arrhythmic drugs (FIG. 3B) lidocaine and isoproterenol (isopro.) treatment (N=3, n=3); FIG. 3C shows Arrhythmic tissues treated with flecainide (N=3, n=3); FIG. 3D shows arrhythmic tissues treated with propranolol (N=3, n=3); FIG. 3E shows arrhythmic tissues treated with amiodarone (N=3, n=3). *p<0.05, **p<0.01, ***p<0.001; and in FIG. 3F panel (i) shows a BrainVision Analyzer 2 snapshot of the electrophysiological profile of day 5 arrhythmic tissues subjected to a single electrical discharge of 2J from a HP Code Master XL⁺ in which the dark regions represent higher Fluo-4 fluorescence, and panel (ii) shows a signal trace of Ca²⁺ fluorescence over time at the region of the tissue indicated by the red box (in panel (i)) in which the distinct stages of the trace are indicated either by a brace symbol or an arrow, where the tissue exhibiting tachycardia is being defibrillated and temporarily arrested, then undergoing initial conduction recovery with periods of ectopic foci firing, and finally resuming in an altered electrical conduction rhythm (Scale bar, 500 μm) and (FIGS. 3A-3F, cell line 1).

FIGS. 4A-4D relate to arrhythmic tissues treated with high concentrations of Methyl-Beta Cyclodextrin (MBCD) showing genomic changes in line with arrhythmia where FIG. 4A shows Gene Set Enrichment Analysis (GSEA) was done on RNA-seq RPKM expression data; FIG. 4B shows KEGG pathway analysis of significant differentially expressed genes where relevant terms and their corresponding −log₁₀(adjusted p-value) are plotted; FIG. 4C shows focused GSEA on the effects of MBCD on cell cycle- and toxicity-related genes; FIG. 4D shows KEGG pathway analysis of significant differentially expressed genes related to toxicity and cellular stress-response, in which, for GSEAs, each node represents a gene set, edges represent gene overlap and nodes are colored based on normalized enrichment score (NES) where a positive NES (Red) denotes enrichment in arrhythmic samples and a negative NES (Blue) denotes enrichment in a control sample with selected clusters being shown and for KEGG pathway analyses, relevant terms and their corresponding -logio(adjusted p-value) are plotted (FIGS. 4A-4D, cell line 1).

FIGS. 5A-5C generally show immunohistochemistry assessment of fibrosis, cell-cell communication and viability in arrhythmic tissues where FIG. 5A shows collagen deposition which was assessed by staining fixed Line 1 microtissues for nuclei (hoechst, blue), collagen I (Col I) and Collagen III (Col III) (green), cardiac troponin T (cTnT; red) where the scale bar represents 100 μm at 20× magnification and the relative expression of Col I & Ill, as well as Col I:III ratio and the passive force in time-matched control and arrhythmic tissues was quantified and expressed in box and whiskers plots (N=3, n=3); FIG. 5B shows relative expression of connexin 43 (Cx43) and N-Cadherin (N-Cad) expression and co-localization which was assessed by staining fixed control and arrhythmic microtissues (N=3, n=3) for nuclei (hoechst, blue), F-actin (phalloidin, magenta) Cx43 (green), and N-Cad (red) where the scale bar represents 50 μm at 60× magnification; and FIG. 5C shows microtissues that were stained for cardiac troponin T (red) and quantification of relative intensity in arrhythmic and control cardiac microtissues (N=3, n=3) where the scale bar represents 50 μm at 60× magnification and the box in the box and whiskers plot extends from the 25^{th} to 75^{th} percentiles, were the whiskers go down to the smallest value and up to the largest value (*p<0.05, **p<0.01, ***p<0.001; FIGS. 5A-5C, cell line 1).

FIGS. 6A-6H generally relate to removal of methyl-β-cyclodextrin mediated arrhythmia reversal where FIG. 6A shows the timeline of assays; FIG. 6B shows representative phase maps of arrhythmic microtissues at day 5 post-treatment with arrhythmia media (top) or with arrhythmic media for 5 days plus control media for an additional 5 days (bottom). Arrows (white) indicate the direction of the wavefront. The legend on the right-hand side details the color associated with specific phase (4, 0, 1, 2, 3, 4) of the calcium transient of a ventricular cardiomyocyte. (Scale bar, 500 μm). FIG. 6C shows representative calcium transient profiles of microtissues treated either the arrhythmic media for 5 days (left) or microtissue treated with arrhythmic media 5 days then control media for 5 days (right) (red scale bar, 1 sec; white scale bar, 500 μm). FIG. 6D shows beating frequency & FIG. 6E shows the contractile force of tissues exposed to constant control media or switched back to control media after 5 days of arrhythmic media (N=3, n=6). FIG. 6F shows passive force arrhythmic tissues on day 5 as well as in arrhythmia reversed tissues on day 10 (N=3, n=6). FIGS. 6G-6H show relative expression of connexin 43 (Cx43) and N-Cadherin (N-Cad) expression and co-localization was assessed by staining fixed control, arrhythmic, and arrhythmia reversal microtissues (N=3, n=3) for nuclei (hoechst, blue), Cx43 (green), and N-Cad (red). The green arrows in the images of FIG. 6G point to Cx43-positive cells. The scale bar represents 50 μm at 60× magnification. The box in the box and whiskers plots of FIGS. 6H extends from the 25^{th} to 75^{th} percentiles, were the whiskers go down to the smallest value and up to the largest value. Line graph represents mean value ±S.E.M. *p<0.05, **p<0.01, ***p<0.001 (FIGS. 6B-6G, cell line 1).

FIGS. 7A-7C generally show an example of a cardiac tissue platform and tissue seeding overview. FIG. 7A shows a rendering of a heart-on-a-chip device with increasing magnification to focus on the dimensions of a single micro-well, as well as the electrical stimulation apparatus where the heart-on-a-chip devices that support the cardiac microtissues were designed using Fusion 360 (Autodesk Canada) and milled from biological inert poly(methyl-methacrylate) (PMMA) or polystyrene using a Tormach mill (Personal CNC 770). Biological inert polydimethylsiloxane (PDMS) (Dow Corning Corporation, 3097358-1004) was mixed at a 1:10 and 1:30 ratio of curing agent to pre-polymer, degassed in a vacuum chamber, and then molded by 27-gauge needles (diameter of 200 μM). The PDMS mixes were cured at 80° C. for 2 hours. The resulting 1:30 and 1:10 rods were placed in pairs horizontally in supporting wells in the PMMA or polystyrene devices to allow for cardiac microtissues to compact between the rods following seeding. The heart-on-a-chip devices that supported the cardiac microtissues were designed using Fusion 360 (Autodesk Canada) and milled from biological inert poly(methyl-methacrylate) (PMMA) or polystyrene using a Tormach mill (Personal CNC 770). Biological inert polydimethylsiloxane (PDMS) (Dow Corning Corporation, 3097358-1004) was mixed at a 1:10 and 1:30 ratio of curing agent to pre-polymer, degassed in a vacuum chamber, and then molded by 27-gauge needles (diameter of 200 μM). The PDMS mixes were cured at 80° C. for 2 hours. The resulting 1:30 and 1:10 rods were placed in pairs horizontally in supporting wells in the PMMA or polystyrene devices to allow for cardiac microtissues to compact between the rods following seeding. FIG. 7B shows cardiac microtissues that were generated with 250,000 hiPSC-derived CMs and human cFibs in a 3:1 ratio in 5mg/mL fibrinogen matrix seeded into the heart-on-a-chip devices. FIG. 7C shows cardiac microtissues (comprised here of iCell hiPSC-CM) naturally compact over time, where compaction plateaus after day 6 post-seeding.

FIG. 8 generally relates to measurements for rod deflection and tissue width at PDMS rods in which representative images of rod deflection at relaxation (left image) and peak cardiomyocyte contraction (right image) in a control sample at day 21 post-seeding are shown. Tissue width at PDMS rods (left-hand image) was measured as the edge-to-edge distance of the tissue construct that is pulling on the 1:30 PDMS rod. Rod deflection for passive force (left-left image) was measured as the distance between the 1:30 PDMS rod in the tissue's relaxed state (upper dashed line), and the rod at non-deflected state (bottom dashed line). For force of contraction (active force), peak rod deflection under electrical stimulation at 1 Hz was measured (upper dashed line). For active force calculations, passive force was subtracted from the force calculated with the maximum deflection.

FIGS. 9A-9D generally relate to preliminary anti-arrhythmic drugs testing results which informed primary electrophysiology and involved performing observational assessments of arrhythmic Line 1 cardiac microtissues following 30 min treatment with a titration series of lidocaine (FIG. 9A), flecainide (FIG. 9B), and propranolol (FIG. 9C) (N=3, n=4). Observational assessments were blinded, were conducted based on brightfield videos of the microtissues and assessed whether the tissues were beating and exhibiting asynchronous (arrhythmic) or synchronous contractions (control). FIG. 9D shows the contractile force of arrhythmic tissues treated with lidocaine (17.5 μg/mL) (N=3, n=4), flecainide (10 μM; N=3, n=4), propranolol (4 μg/mL; N=3, n=4).

FIGS. 10A-10B generally show High MBCD treatment-mediated protein-level changes to cell death and Brain Natriuretic Peptide (BNP) where FIG. 10A shows cell death which was assessed by staining fixed Line 1 microtissues (3N, 3n) for nuclei (hoechst, blue), TUNEL (fluorometric dye, green), and cardiac troponin T (red). The scale bar represents 100 μm at 20× magnification. FIG. 10B shows secreted BNP (pg mL⁻¹) from Line 1 microtissues on day 5. The box in the box and whiskers plot extends from the 25th to 75th percentiles, where the whiskers go down to the smallest value and up to the largest value (“*” represents p<0.05, “**” represents p<0.01, and “***” represents p<0.001).

FIGS. 11A-11F generally show MBCD-induced arrhythmias in a second hPSC-CM line where FIG. 11A shows the Percentage (%) of microtissues treated with control (CTRL) and arrhythmic (Arr) media conditions exhibiting arrhythmic or non-arrhythmic phenotypes; FIG. 11B shows force of contraction (mN mm⁻²) and beating frequency (BPM) of Line 2 CTRL and Arr tissues over the treatment regimen (N=3, n=4); FIG. 11C shows phase maps of a Line 2 HES3-NKX2.5 arrhythmic tissue assessed on day 5 of MBCD-FA treatment, where each image is taken 10 frames apart, the legend on the left-hand side details the color associated with specific action potential phase and the red scale bar represents 500 μm. FIG. 11D shows Mean wavefronts per wave (N=3, n=4); FIG. 11E shows conduction velocity (cm/s; N=3, n=4); and FIG. 11F shows cycle length (ms; N=3, n=4) for day 5 Line 1 and 2 microtissues exposed to either control or arrhythmogenic media conditions (where “*” represents p<0.05, “**” represents p<0.01, and “***” represents p<0.001). The box in the box and whiskers plot extends from the 25^{th} to 75^{th} percentiles, where the whiskers go down to the smallest value and up to the largest value (FIGS. 11A, 11D-11F, cell line 1 & 2; FIGS. 11B-11C, cell line 2).

FIGS. 12A-12B generally show electrophysiological characterization of cardiac microtissues on Days 0, 4, & 5 of control or arrhythmogenic treatment. The tissue phenotype was categorized by the number of wavefronts present as well as the directionality of these wavefronts. The prevalence of specific phenotypes was presented as a percentage (%) of the total tissues exposed to either control or arrhythmogenic media on the specific day during which the tissue's electrophysiology was assessed. The images marked by * are isochrone maps of the same tissue but taken in sequence. This allows for the identification of 2 separate excitatory foci, where one impulse originates on the right-side of the microtissue and move to the left (left-hand image) and the other depicts an activation from the bottom to the top of the microtissue (right-hand image). The images marked by ** show that for the tissues categorized under 2-Multiple wavefronts (R-entry and Ectopic Foci), regular isochrones could not be generated because there was never a period without a wavefront. Therefore, a MATLAB™ program was adjusted to take this into account; however, instead of using the colors to represent conduction speed, the colors represent the phase of the Ca²⁺ transient at that point, with the black lines indicating where distinct wavefronts are present.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein.

Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical signal, electrical connection, or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

In addition, it should be noted that the phrases “at least one of X and Y” or “X, Y or a combination thereof” is intended to mean X, Y or X and Y.

It should also be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term that it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

In one aspect, in accordance with the teachings herein, there is provided a model that incorporates the cell membrane and Ca²⁺-handling disruption properties of methyl-β-cyclodextrin (MBCD) [13-15] and heart-on-a-chip technology to model acquired cardiac arrhythmias in a human-relevant system. For example, in at least one embodiment, the model is a 3D human model of cardiac arrhythmia on a microchip setup with high reproducibility and fidelity, and extensive functional applicability. In one example embodiment, to mimic in vivo conditions, the 3D human model involves the combination of human induced pluripotent stem cell-derived cardiomyocytes (hiPSC-CMs) or human embryonic stem cell-derived cardiomyocytes and cardiac fibroblasts from healthy controls in a biocompatible fibrin hydrogel which are then seeded between two deflectable polymeric rods.

In another aspect, the functional properties of an example embodiment of the 3D human model along with novel optical imaging techniques were used to demonstrate dramatic changes in contraction rate, synchronicity, and electrophysiological conduction in arrhythmic tissues relative to controls. Taken together, these data demonstrate the distinctive potential of the 3D human model defined in accordance with the teachings herein for pathophysiological studies (assessing contractility and 3D spatial conduction), and for arrhythmia drug testing applications.

For example, in at least one embodiment, human 3D microtissues were generated by seeding hiPSC-CMs and cardiac fibroblasts in hydrogels into microwells designed to enable active and passive force assessment. Calcium signaling was disrupted using methyl-beta cyclodextrin (MBCD), which has been shown to disassemble cell-cell gap junctions. The experimental data included herein shows that resulting arrhythmias were progressive and present in all microtissues within 5 days of treatment. The experimental data also showed that arrhythmic tissues exhibited reduced conduction velocity and an increased number of distinct action potentials and action potential cycle length as well as a significant reduction in contractile force generation, an initial increase in beating frequency, and increased passive force and collagen deposition in line with fibrosis. In another aspect, a subset of tissues with more complex arrhythmias exhibited spatial differences in wave propagation in 3D. Pharmacological and electrical defibrillation was successful. Transcriptomic data indicated an enrichment of genes consistent with cardiac arrhythmia. MBCD removal reversed arrhythmias resulting in synchronicity despite not resolving fibrosis. This novel human-relevant 3D model of arrhythmia shows potential for improving the understanding of conduction propagation in arrhythmias and for the development of new therapies.

Taking the experimental data together, the 3D human model as described in accordance with the teachings herein exhibits one or more of the following characteristics: 1) a reliable and easy-to-follow methodology to generate arrhythmias of increased complexity with increased treatment duration; 2) complex arrhythmias that exhibit multiple wavefronts at a given time point, which is a hallmark of complex arrhythmias; 3) arrhythmic tissues that showed for the first time 3D (i.e. X-, Y, and Z-axis) differential effects in wave front migration; 4) sustainability of arrhythmias allowing the assessment of arrhythmia initiation, maintenance and termination; 5) a classic response to MBCD resulting in alterations in gap junctions and wave propagation; 6) converting from an arrhythmic state with established pharmacological agents and electrical cardioversion; and 7) the ability to display sustainable acquired arrhythmias faster than other models (-10 days vs. 30 days for optical tachypacing [9]) without the requirement for genetic modification of the cell line, use of optogenetic stimulation apparatus or electrical stimulation. Combined, these properties mimic the pathophysiological properties of arrhythmias in actual hearts.

### Methods and Materials

### Heart-on-a-Chip Device Preparation & Cell Seeding

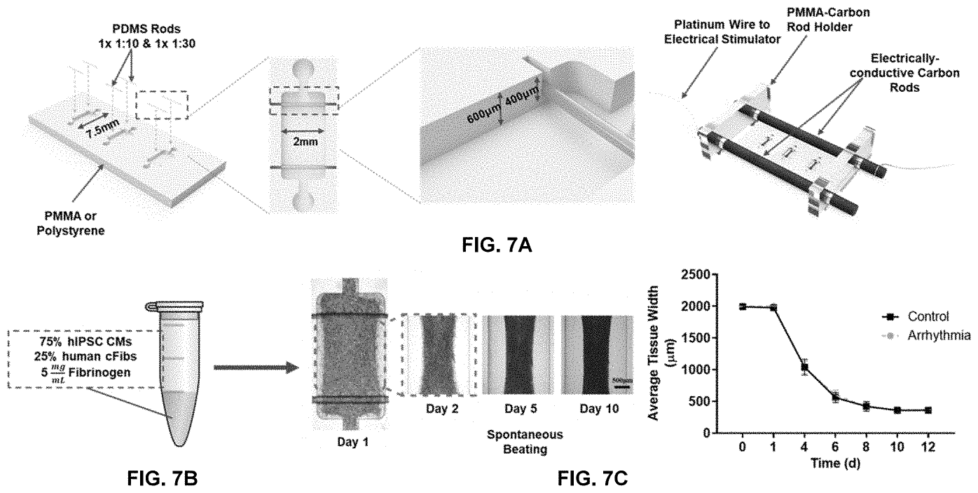

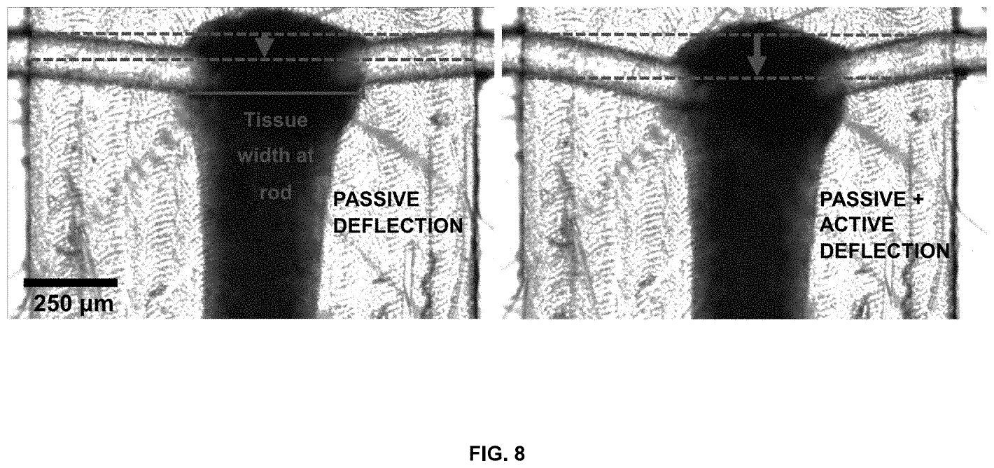

The heart-on-a-chip used for the 3D human model may be designed and fabricated as represented in FIGS. 7A-7C with a methodology and characterization described in Mastikhina et al. (2019) [16]. Briefly, 2 different hPSC-CM lines were tested: 1) an induced pluripotent stem cell-cardiomyocytes cell line (hiPSC-CM, referred to as cell line 1; shown in Table 1; iCell Cardiomyocytes; Cellular Dynamics; female sex) and 2) an embryonic stem cell-cardiomyocyte cell line (cell line 2: hESC HESS-NKX2-5eGFP/w, referred to as hESC-CMs; WiCell Research Institute) which was provided by Dr. Gordon Keller. The hPSC-CMs and human cardiac fibroblasts (Lonza; lots #0000493460 and 0000565615; female sex) were seeded in a 3:1 cell ratio (250,000 cells/well) to reflect the overall volumetric ratio of CMs to non-CMs in the human heart [19]. In alternative embodiments, the aforementioned cell ratio may range from about 1:1 to about 9:1 but in some cases, it is preferable for the ratio to be about a 3:1 cell ratio. The cells were homogeneously mixed into a fibrin mixture consisting of fibrinogen (5 mg/mL; Millipore Sigma; F3680), aprotinin (0.00825 mg/mL), Y-27632 (10 nm, STEMCELL; 72304), thrombin (2 U/mL; Sigma-Aldrich), Matrigel (1 mg/mL; Corning; 354230), and DMEM (1×) (see FIG. 7B). Using cold pipettes, the cell-fibrin mixture was quickly seeded into the heart-on-a-chip wells (250,000 cells/well) to prevent premature solidification (e.g., once the enzyme that converts fibrinogen into fibrin is added it works by forming a gel and seeding is performed in a period of time which may be referred to as a seeding time, which may be within a range of about 5 seconds up to about 45 seconds, to prevent the gel from polymerizing into the pipette tip). Referring now to FIG. 7C, iCell plating media (iCell Cellular Dynamics) with Antibiotic-Antimycotic (1×; Gibco) was used for 48 h to allow for tissues settlement and compaction. After 48 h, the plating media was changed to StemPro-34 Serum Free Media (ThermoFisher) supplemented with L-glutamine (2 mM), L-ascorbic acid (50 μg/mL), transferrin, 1-thioglycerol (0.039 μL/mL, Sigma-Aldrich), and antibiotic-antimycotic. The media was replaced every second day throughout the tissue remodeling phase (Day −7 to 0). In various embodiments, the composition of the media may vary such as, but not limited to, including serum, including different ranges of serum and excluding serum, for example.

It should be noted that in an alternative embodiment, other cell types may be used in the 3D human heart model such as, but not limited to, the addition of endothelial cells, and/or cardiomyocytes of the conduction system such as nodal cells, cardiomyocytes of an atrial phenotype or other cardiomyocyte subtypes, which will allow for the creation of more complex and/or chamber-specific tissues. For example, these cells may be added to the hydrogels at the time of seeding. These alternative 3D human heart models may also be treated with MBCD to study potential arrhythmias.

TABLE 1
Background information on cardiomyocyte cell lines
used. Cell lines were categorized by company, catalog
number, lot number and patient demographics.

| | | Company/ | Catalog | |
|---|---|---|---|---|
| Line | Cells | Source | # | Patient Demographics |
| 1 | iCell | Fujifilm | c1006 | Female; Age: <18; |
| | Cardiomyocytes | Cellular | | Caucasian; Tissue |
| | (cell line A) | Dynamics | | Source: Fibroblast; |
| | | Inc. | | Reprogramming Method: |
| | | | | Retroviral Transduction |
| 2 | HES3-NKX2.5 | Dr. | N/A | Female; 54% |
| | (cell line B) | Gordon | | ventricular-, 24% |
| | | Keller | | atrial-, and 22% |
| | | | | nodal-like CMs |
| N/A | Human | Lonza | CC- | Female; Age 60 |
| | Ventricular | | 2904 | |
| | Cardiac | | | |
| | Fibroblasts | | | |

### Arrhythmia Induction and Reversal

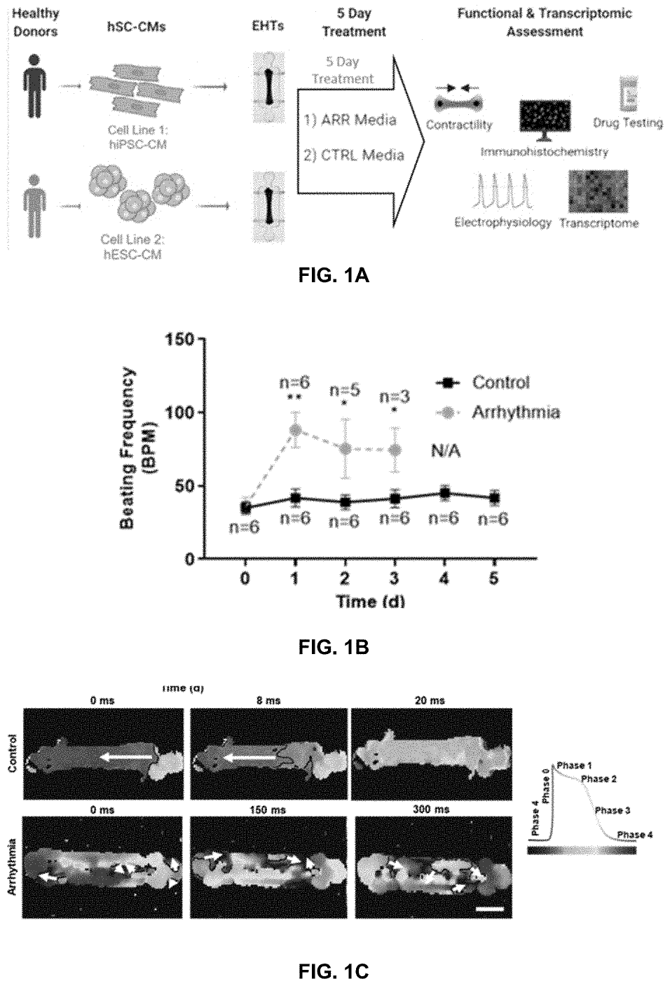

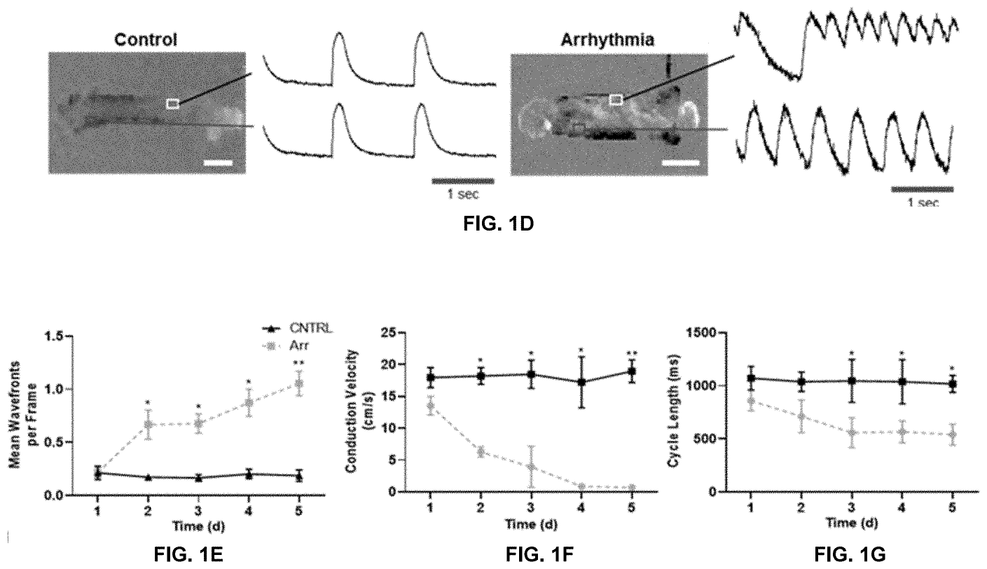

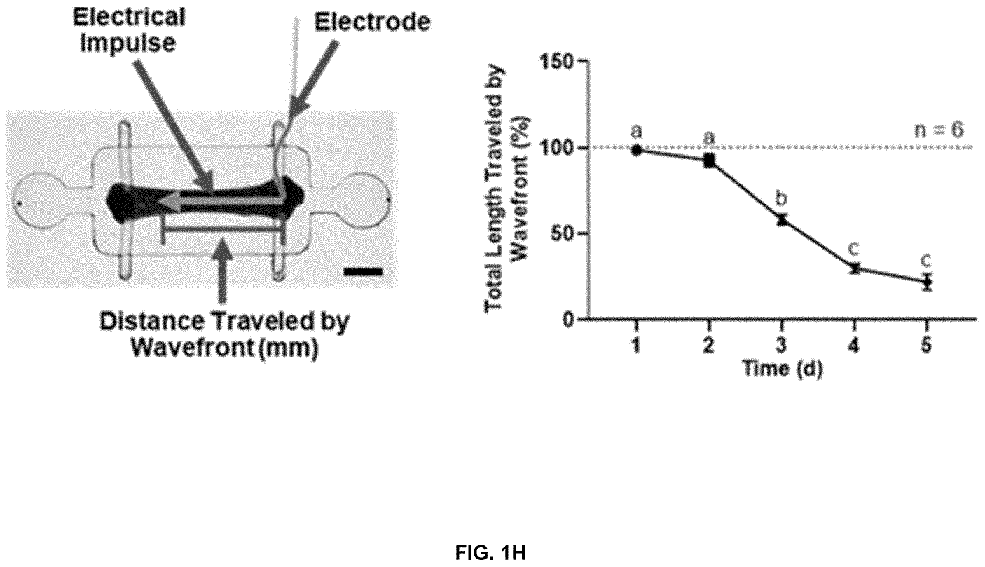

Tissues were allowed to compact for 7 days (Day -7 to 0) and treatment with either arrhythmogenic or control media conditions began on day 8 post-seeding (e.g. Day 0 in FIG. 1A). Arrhythmogenic media consisted of DMEM no glucose (ThermoFisher; 11966025), B-27 minus insulin supplement (1×; ThermoFisher; A1895602), HEPES (10 mM), L-carnitine (2 mM), creatine (5 mM), taurine (5 mM), MEM non-essential amino acids (1033 ; ThermoFisher; 11140050), insulin-transferrin-selenium (1×; ThermoFisher; 41400045), galactose (10 mM), MBCD or MBCD-conjugated FAs as follows: hiPSC-CMs were treated with 100 μM FAs (linoleic acid 40 μM, oleic acid 40 μM, and palmitic acid 20 μM; MBCD 0.8 mM); and hESC-CMs were treated with 200 μM FAs (linoleic acid 80 μM, oleic acid 80 μM, and palmitic acid 40 μM; MBCD 1.6 mM)), glutamine (1 mM), and antibiotic-antimycotic. Control medias did not contain galactose or MBCD-FAs. Instead, control media were supplemented with glucose (5 mM) and either FA vehicles (MBCD) at a lower dose of MBCD than the arrhythmogenic media (0.5 mM) or no MBCD (to confirm MBCD was the arrhythmogenic agent). Microtissues were treated by addition of media containing the components described above for up to 5 days and analyzed (e.g., imaging, tissue harvesting for isolation of RNA for transcriptomic analysis, fixing tissues for histopathological assessments/staining) at different time points to assess functional and transcriptomic parameters (see FIG. 1A).

It should be noted that while there were other components described for the arrhythmogenic media, the inventors determined that by performing various tests in which there were substitutions made for these components as well as eliminating MBCD, that it was MBCD which was the main driver of arrhythmia in the 3D human model. However, it is possible that some of the components of the media described above (e.g., fatty acids) may potentialize the actions of MBCD.

### Tissue Contractile Force, Spontaneous Beat Rate, and Compaction Measurements

For electrical stimulation, the devices (e.g., chips) with the cardiac microtissues were placed between two parallel carbon rods (Ladd Research, 30250) spaced 1.5 cm apart inside a petri dish, each with a diameter of 3mm. Platinum wire (Ladd Research, 30571), which is a biocompatible material, was securely wrapped around each carbon rod and extended to the outside of the petri dish (see FIG. 7A). Each platinum wire was then connected through test leads with alligator clips to a pulsed electric stimulator (Astro-Med Grass S88X Stimulator). Other setups may be used for the chips in alternative embodiments, by varying the dimensions and/or placement of the above-noted elements.

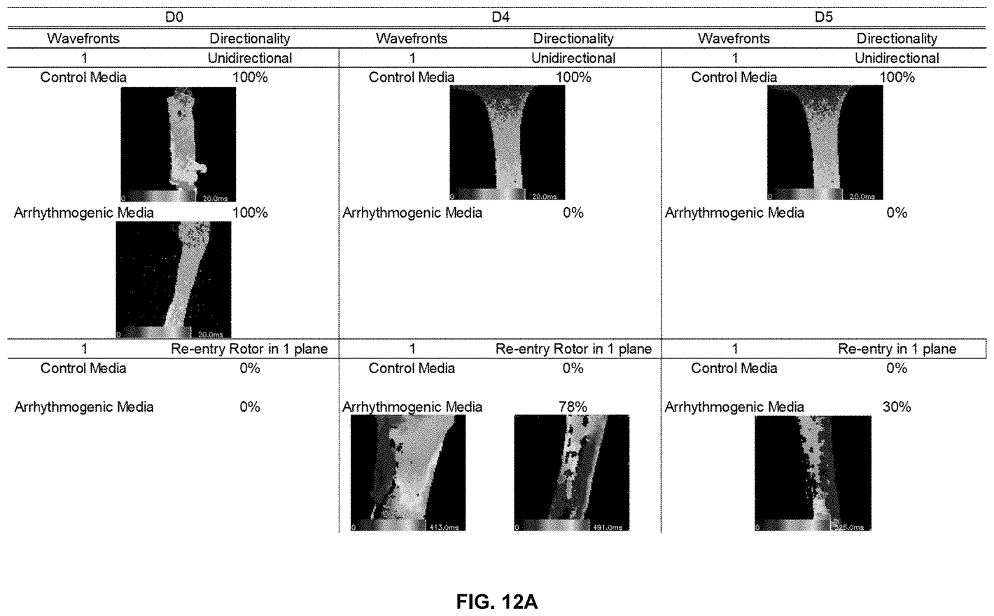

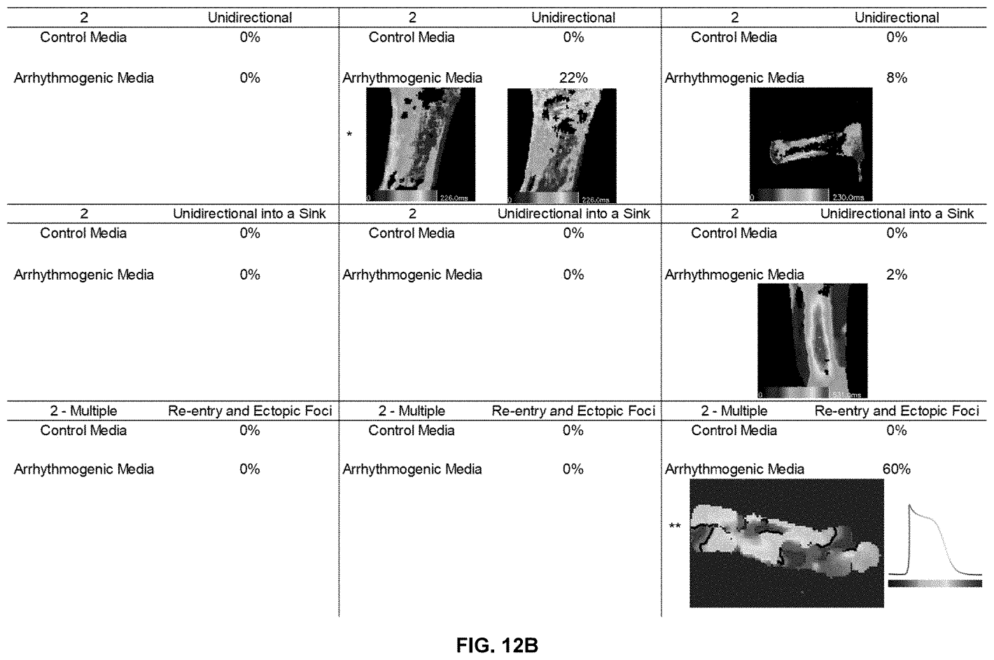

During contractile force measurements, cardiac microtissues were electrically stimulated at 1 Hz and at their respective excitation threshold and videos of the 1:30 PDMS rods were taken for 15 seconds under 10× magnification with a Leica EC3 camera. From these videos, rod displacement and tissue width was measured and is shown in FIGS. 12A-12B. For tissue compaction and spontaneous beating rate measurements, the tissues were not stimulated.

The following force vs. displacement equation [16] was used to assess the contractile force generated by the microtissues:

$$Active Contractile Force ( nN {mm}^{- 2} ) = \frac{\begin{matrix}( ( 0.002156 * y * ( {x}_{a} + {x}_{p} ) ) + ( 0.00256 * {( {x}_{a} + {x}_{p} )}^{2} ) + \\ ( 1.55 * ( {x}_{a} + {x}_{p} ) ) - ( 0.002 \; 56 * {x}_{p}^{2} ) - ( 0.002156 * y * {x}_{p} ) - ( 1.55 * {x}_{p} ) )\end{matrix}}{C S A}$$

where xₚ=passive displacement, xₐ=active displacement, y=width, and CSA=cross-sectional area (mm²).

As tissues became arrhythmic it was not possible to accurately measure rod displacement due to lack of synchronicity and lack of response to electrical stimulus (e.g., lack of capture, as illustrated in FIG. 1H). Therefore, these were not included in force measurement analyses.

### Brain Natriuretic Peptide (BNP) Secretion Quantification

Culture media was collected on day 5 of arrhythmia induction. Human BNP levels were measured using an ELISA kit (Abcam) as recommended by the manufacturer.

### Optical Mapping

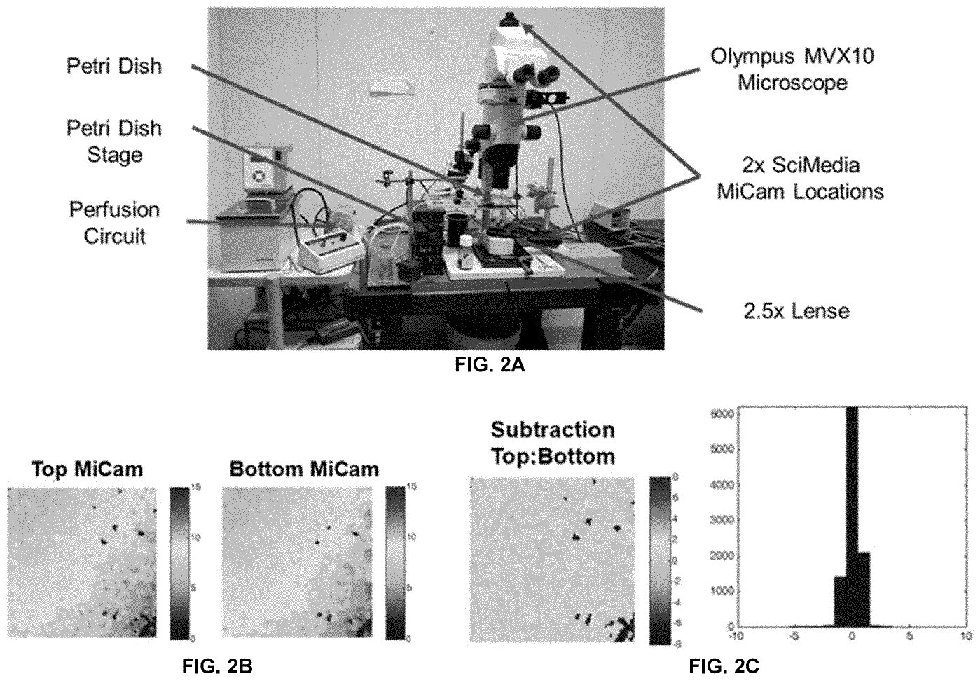

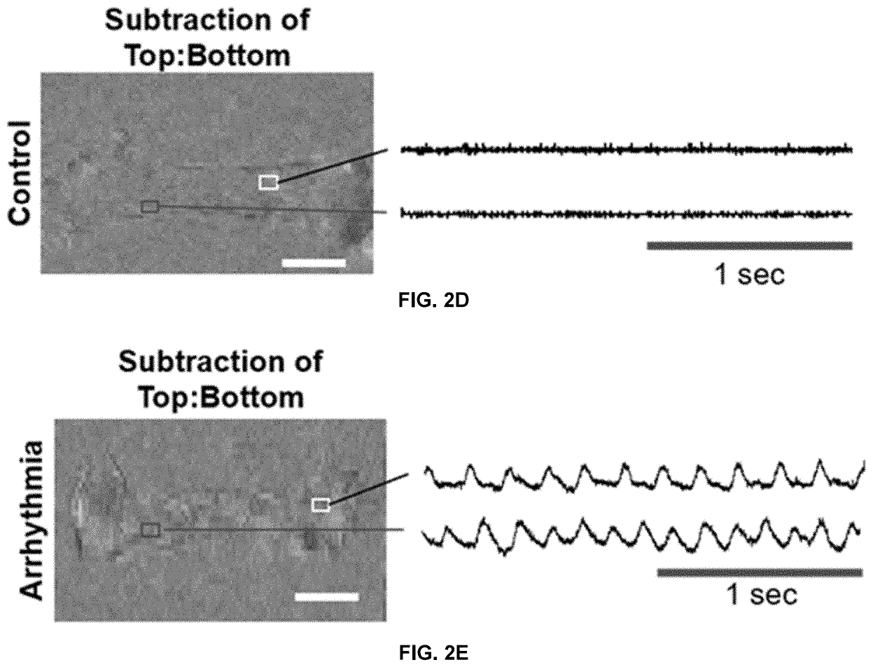

To characterize arrhythmic phenotypes, Ca²⁺ dynamics were assessed using the calcium indicator Fluo-4 (0.5pg in 1% DMSO-culture media; ThermoFisher). The experimental electrophysiology setup for performing this assessment and measurement of wave propagation in 3D is depicted in FIG. 2A. Fluorescent signals were measured using a MVX10 Fluorescence MacroZoom Upright Microscope (Olympus Science) with two Ultima-L (SciMedia) optical cameras positioned above and below the microtissue (i.e., to allow the top and bottom surfaces of the microtissue to be imaged at the same time). The two cameras were aligned and set to capture images simultaneously by following the manufacturer's instructions. To enure camera synchronicity, this setup was verified using a monolayer of cardiomyocytes (FIGS. 2B-2C). Electrophysiological results were analyzed using BrainVision Analyzer 2 (Scimedia). BrainVision allowed the creation of videos of calcium wave fronts as observed from the two cameras depicting the top and bottom of the cardiac microtissues. Isochrone maps were generated using a program written in IDL (RSI) (see Williams et al., “A 3-D human model of complex cardiac arrhythmias”, Acta Biomaterialia, Vol. 132, Sep. 15, 20221, pages 149-161, which is hereby incorporated by reference in its entirety). Due to current limitations in isochrones mapping software, extremely complex arrhythmias such as re-entry rotors (e.g., see FIG. 13B, column D5, final row) could not be converted to isochrone maps. Therefore, an in house software program [20] that uses phase mapping was used to quantify the complexity of the arrhythmias seen on day 5.

To assess the range of stimulation capture, microtissues were stimulated by a silver electrode at 3 Hz and 6V, and the distance from the electrode to which point the cardiac microtissue was not electrically captured by the delivered electrical impulse was marked as the range of stimulation capture (mm) (e.g., see FIG. 1H).

### Anti-Arrhythmic Drug Testing & Electrical Defibrillation

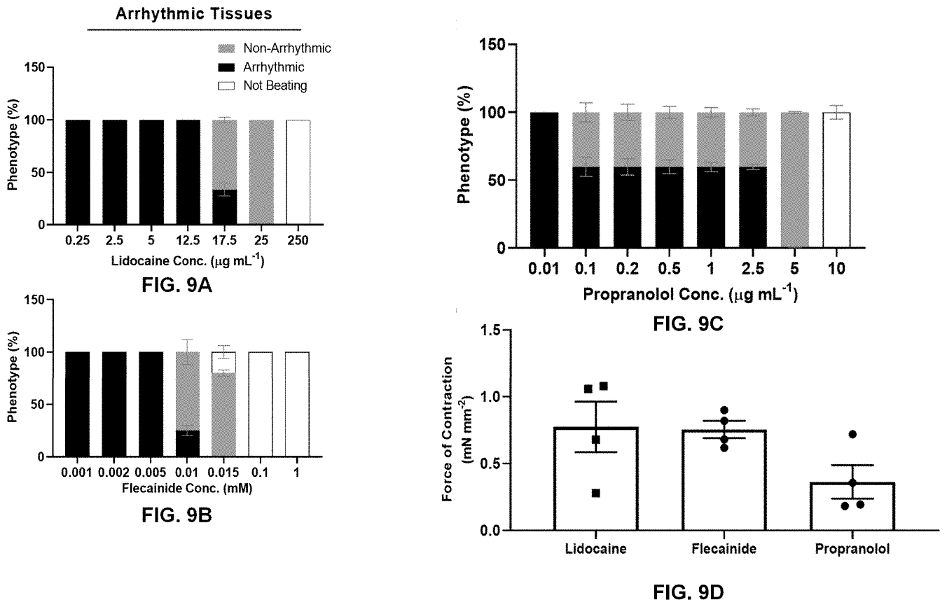

Arrhythmic tissues were treated with a Class IB (Lidocaine HCl, 17.5 μg/mL, Teligent), IC (Flecainide, 10 μM, Sigma-Aldrich), II (Propranolol, 4 μg/mL, Sandoz), or III (Amiodarone 30 μg/mL, Fresenius Kabi) anti-arrhythmic agents for 30 mins. The anti-arrhythmic drug concentrations were determined based on the visual and contractile force measurements (see FIGS. 9A-9D). For defibrillation experiments, platinum wires were connected by alligator clips to a modified HP Code Master XL⁺ defibrillation unit. The defibrillation impulse was set at 2J.

### Immunohistochemistry

Cardiac microtissues were fixed on day 5 of treatment with 4% PFA overnight at 4° C. prior to being washed with Phosphate Buffered Saline (PBS). Immunostaining was performed on whole tissues using the following antibodies: mouse anti-cardiac troponin (1:100, Thermofisher; MS-295-P1), mouse anti-N-cadherin (1:250, Thermofisher; 33-3900), donkey anti-mouse Alexa Fluor 647 (1:500, Thermofisher; A31571), rabbit anti-connexin 43 (1:250, Abcam; ab11370), goat anti-rabbit Alexa Fluor 568 (1:500, Thermofisher; A11011), rabbit anti-vimentin (1:100, Cell Signaling Technology; 5741), mouse a-smooth muscle actin (1:250, Sigma-Aldrich; A2547), F-actin Phalloidin Alexa Fluor 488 (1:50, Thermofisher; A12379), goat anti-collagen type I (1:250, Southern Biotech; 1310-01), goat anti-collagen type III (1:250, Southern Biotech; 1330-01), donkey anti-mouse Alexa Fluor 647 (1:500, Thermofisher; A31571), goat anti-rabbit Alexa Fluor 568 (1:500, Thermofisher; A11011), rabbit anti-goat Alexa Fluor 488 (1:500, Thermofisher; A11078). Cell viability was accessed with the DeadEnd™ Fluorometric TUNEL System (Promega; G3250). Tissues were imaged using a fluorescence confocal microscope (Zeiss LSM-510). The viability of the cell lines in cardiac microtissues over an extended periods has been assessed previously [16-18].

### RNA Sequencing

RNA was extracted from control and arrhythmic cardiac microtissues on day 5 (N=3). RNA-seq counts were analyzed on Partek® Flow®^{[18]}. Reads were aligned to the human reference genome assembly GRCh38 using STAR. Quantification to the annotation model was performed using Partek E/M. 1 count was added to all gene expression values prior to RPKM (reads-per-kilobase per million) normalization. Differential expression analysis was done using the Gene Specific Analysis (GSA) method on Partek. Pathway analysis was performed on significant genes (p-value<0.05) using g:Profiler's g:GOSt tool with the Kyoto Encyclopedia of Genes and Genomes (KEGG) database (Release 96.0, Oct. 1, 2020). Gene set enrichment analysis (GSEA, ver. 4.1.0) was carried out on RPKM normalized expression dataset [23]. The Bader lab's Enrichment Map, Human_GOBP_AllPathways_no_GO_iea_Mar._01_2020_symbol.gmt gene set was used for analysis [24]. Gene sets containing greater than 500 genes and less than 15 genes were excluded. Genes were ranked based on GSEA “Signal2Noise” metric and 1000 permutations were carried out using the “classic” enrichment statistic setting. GSEA output was visualized on Cytoscap (ver. 3.8.1) [25] using Enrichment Map (ver 3.3) [24]. Nodes and edges were filtered based on p-value (<0.001) and FDR q-value (<0.05). Nodes were further filtered based on a normalized enrichment score (NES) of greater than +2 and less than −2. Relevant nodes were visualized, and cluster identities were assigned manually.

### Statistical Analysis

Each statistical analysis was completed on datasets consisting of at least N=3 (biological replicates) with at least n=2 (technical replicates). Statistical analysis was done with GraphPad Prism 7 & 8 (Graph Pad Software, Inc.). Repeated Measures 1-way ANOVAs were used to analyze tissue beating frequency and force measurements, and Student's t-tests were conducted on datasets with only two parameters. A p value below 0.05 was considered significant.

### Results

### Arrhythmia Induction and Characterization

Cardiac microtissues generated with two different cell lines (FIG. 1A, human iPSC- or embryonic stem cell-derived CMs) were seeded at 3:1 ratio of CMs to human cardiac fibroblasts in a fibrin hydrogel to reflect the volumetric ratio of CMs to non-CMs in the human heart [19]. While a 3:1 ratio has been found to be the best ratio for tissue stability and contractility in various cases, it is also possible that other ratios may be used in other embodiments such as about 1:1 up to about 9:1. To that end, cells were seeded in a previously described heart on-a chip device with real-time functional measurements capabilities (see FIGS. 7A-7C) [16]. Cells were allowed to remodel the hydrogel for 7 days, as previously described [16], forming compacted tissues of 2.0×0.5×0.4 mm dimensions (L×W×D) that beat synchronously [16]. However, in alternative embodiments the arrhythmogenic media may be added to the cardiac tissue about five to nine days after the seeding is performed, with a period of 7 days being preferable in some cases.

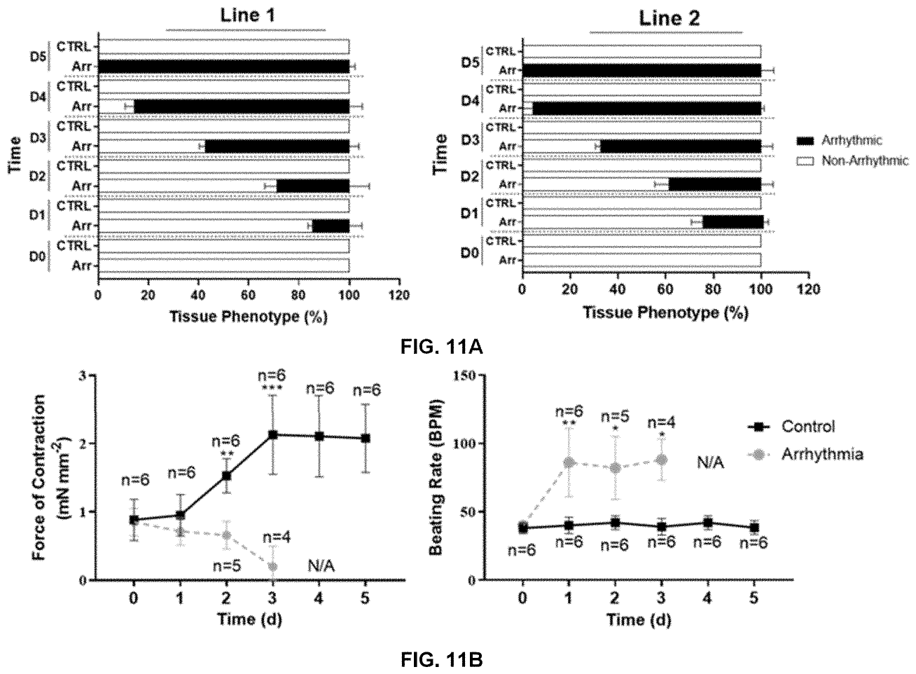

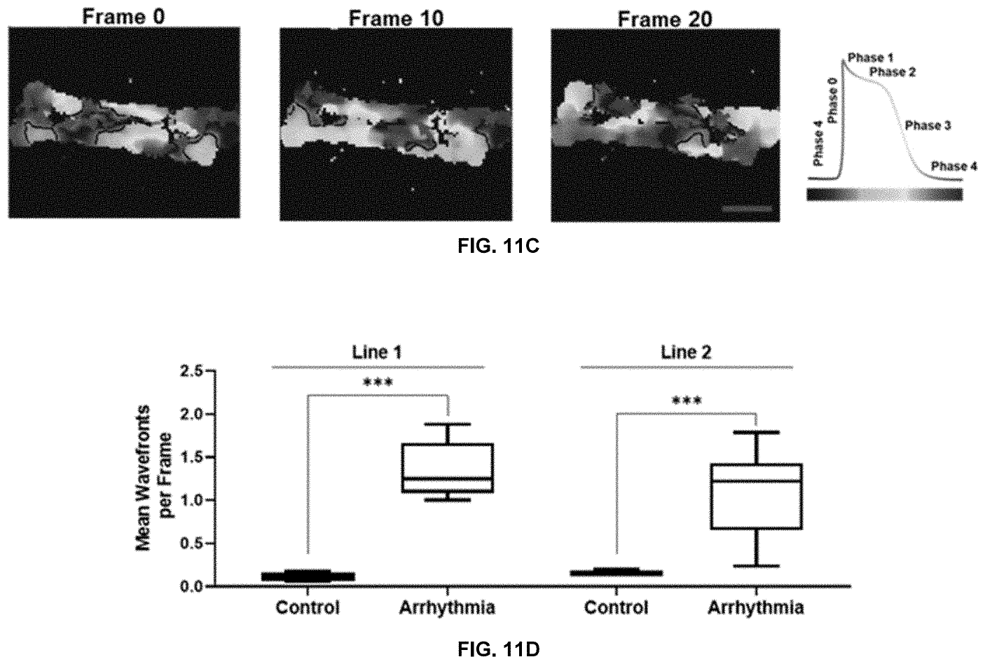

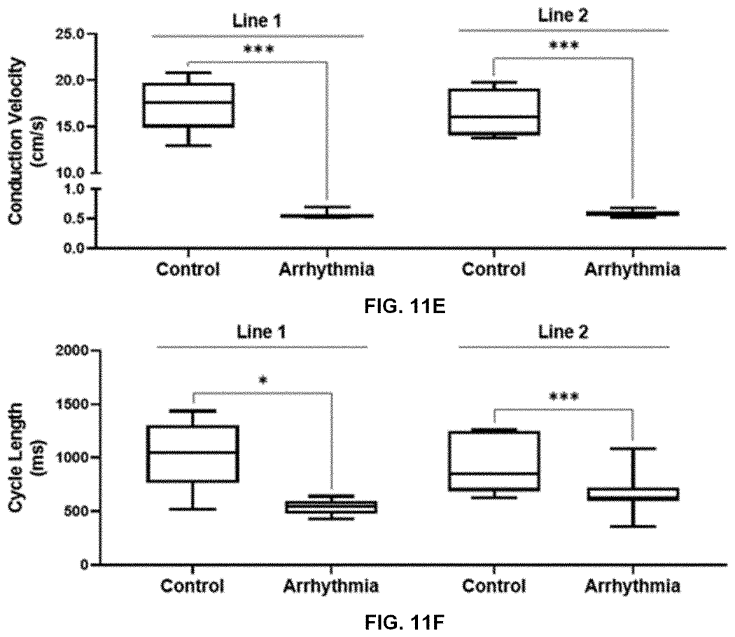

To induce arrhythmia, tissues were treated with media containing a high concentration of MBCD (e.g., such as about 0.8 mM MBCD or higher; henceforth referred to as arrhythmogenic media). Tissues treated with both MBCD-free and low MBCD concentration (0.5 mM MBCD) media, henceforth referred to as control media, did not develop arrhythmias and beat in synchrony for the entire length of the treatment regimen (5 days). Conversely, tissues treated with arrhythmogenic media exhibited a significant increase in the spontaneous beating frequency (as early as 24 h post-treatment) (see FIG. 1B, p<0.001) and progressively increased arrhythmias, whereby within 5 days of treatment all arrhythmogenic media-treated tissues exhibited a complex arrhythmic phenotype. This was characterized via optical mapping in which the tissues exhibited multiple wave fronts and complex migration patterns compared to controls (see FIGS. 1C-1D, FIGS. 12A-12B and FIGS. 11C-11D). Of note, it was not possible to assess spontaneous beating rates at later time points given the increasingly erratic behavior of the tissues (see FIG. 1B).

To quantify the electrophysiological complexity of the arrhythmic tissues, the mean number of wave fronts per frame was assessed via optical mapping. When compared to time-matched controls, arrhythmic tissues exhibited a time-dependent, significant increase in the mean number of wave fronts over time (see FIG. 1E) with a corresponding progressive reduction in conduction velocity (see FIG. 1F) and shorter cycle lengths (see FIG. 1G). Moreover, arrhythmic tissues at day 5 were not paced (e.g., did not respond to electrical stimulation) and did not generate synchronous contraction, exhibiting instead a fibrillatory-like behavior. Tissues treated with arrhythmogenic media showed a time-dependent, progressive, and significant reduction in the distance of wave propagation range compared to time-matched controls and arrhythmic tissues at day 1 post-treatment (see FIG. 1H). This lack of capture of the electrical stimulus is a hallmark of arrhythmias in the clinic.

To investigate if the complex arrhythmias observed on day 5 showed differential effects in 3D, an electrophysiology set up was created that allowed imaging of the top and bottom surfaces of the 3D tissues simultaneously. This set up involved positioning 2 high speed cameras above and below the field of view to allow for a 3D representation of the tissue (see FIG. 2A). To validate camera synchronicity, monolayers of cardiomyocytes were imaged and the signal subtraction of the acquired image data from the top and bottom cameras was plotted (see FIG. 2B), which confirmed alignment. Analysis of control tissues in this dual camera setup, showed no differential wave propagation in 3D in any of the time points tested, as seen by the flat line of subtracted signals (see FIGS. 2D-2E), characteristic of the different time points. However, 3D differences were observed in 14.3±2.6% of arrhythmic tissues at day 5, as evidenced by the differences in wave propagation signal between top and bottom surfaces (see FIGS. 2D-2E; arrhythmic tissues N=3, n=7; control tissues N=3, n=3).

### Successful Pharmacological & Electrical Cardioversion of Arrhythmic Tissues

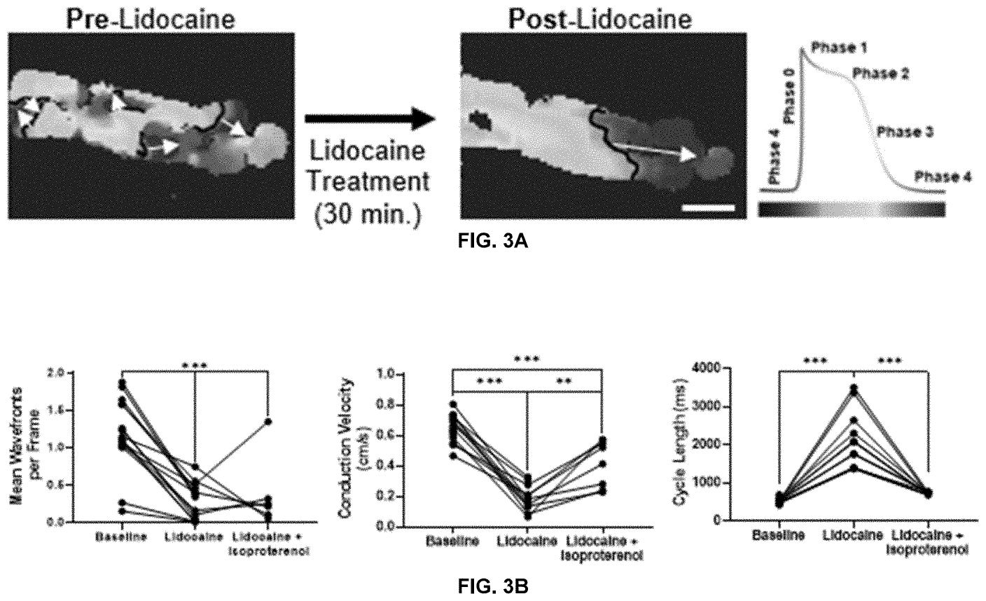

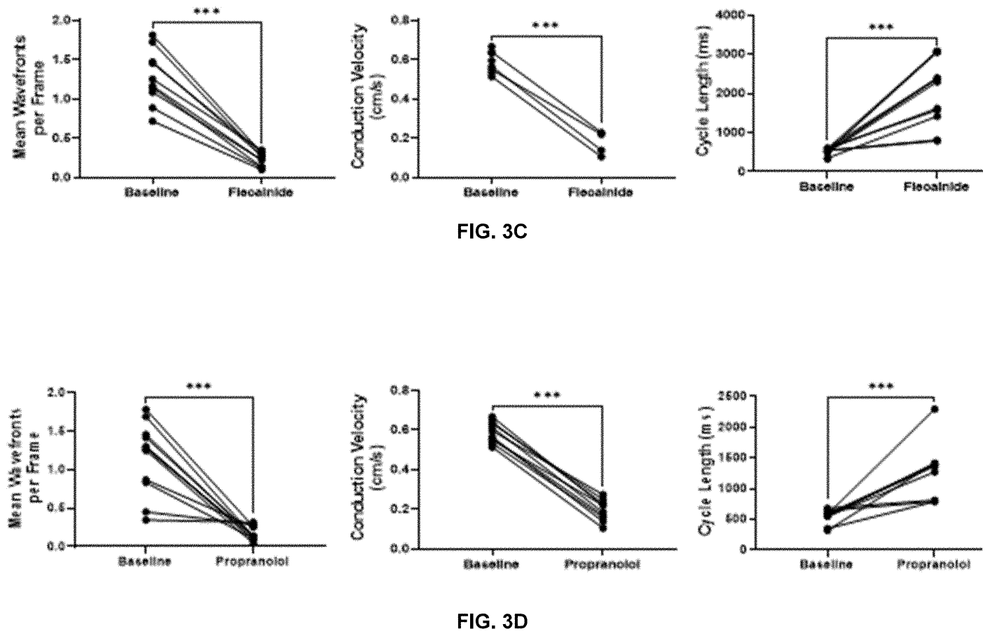

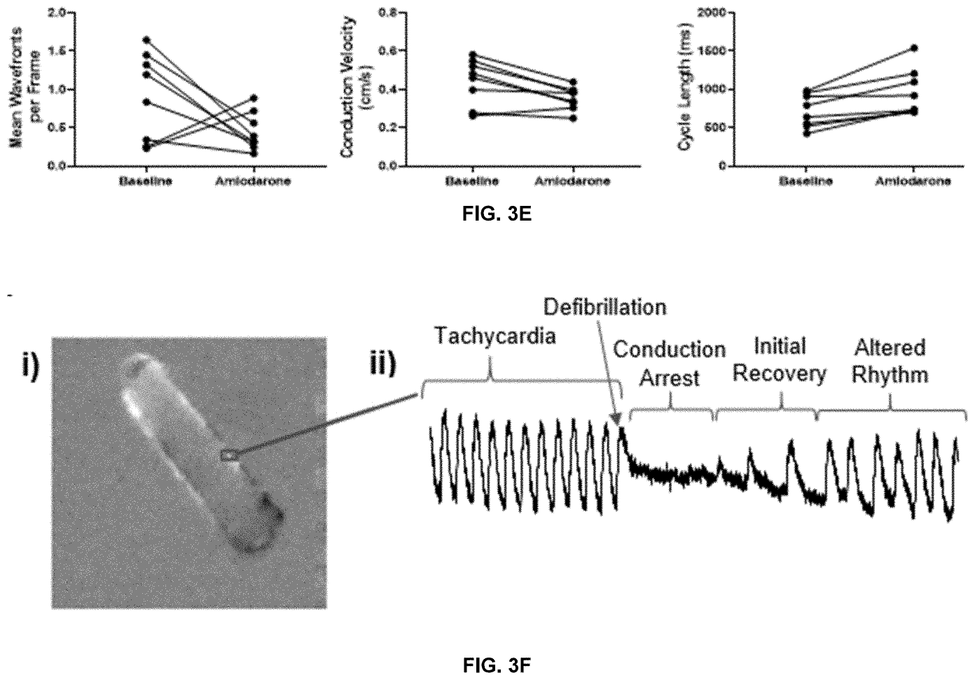

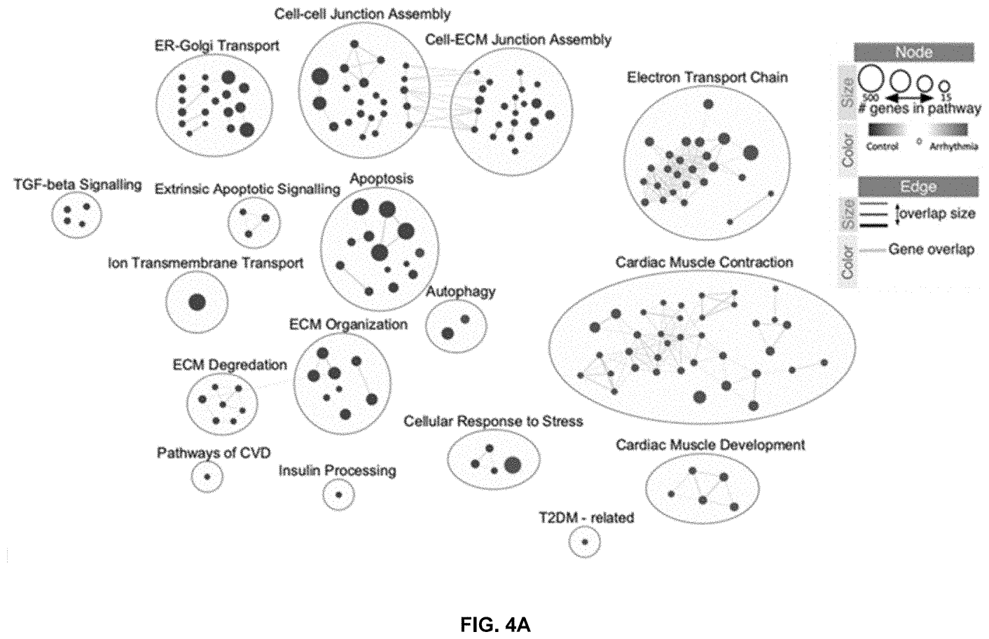

Given the sustained nature of the arrhythmias observed, it allowed testing of pharmacological and electrical interventions in arrhythmia termination. First, dose-response curves for the different drugs were generated (see FIGS. 9A-9D) and the lowest concentration with the highest effect was chosen for in depth analysis. Acute treatment of arrhythmic tissues with lidocaine (Class IB/C agent) resulted in a significant reduction in the mean number of wavefronts per frame (see FIGS. 3A-3B) to levels similar to those of control tissues (see FIG. 1E). Lidocaine treatment also significantly decreased conduction velocity while concomitantly increasing cycle length duration (see FIG. 3B). The addition of isoproterenol to lidocaine-treated tissues resulted in no change to mean wavefronts per frame, and an increase in conduction velocity, and a reduction in cycle length. This confirmed that the effects of lidocaine were not the result of cell death, as the addition of isoproterenol to lidocaine treated tissues could reverse conduction velocity and cycle length depression. Acute treatment of arrhythmic tissues with flecainide (Class IB/C agent, see FIG. 3C) and propranolol (Class II agent, see FIG. 3D) resulted in similar electrophysiological changes as lidocaine (see FIG. 3B). The resulting synchronicity achieved with treatment of arrhythmic tissues with anti-arrhythmic drugs allowed assessment of force of contraction, showing that drug treatment led to a gain in function (see FIG. 9D). However, treatment with amiodarone (Class III agent) did not produce any significant electrophysiological changes (see FIG. 3E). A defibrillation with an impulse of 2J was sufficient to arrest the tachycardic electrical conduction fora period of time (see FIG. 3F).

### Transcriptomic Profile of Arrhythmic Tissues is in Line With Calcium Overload

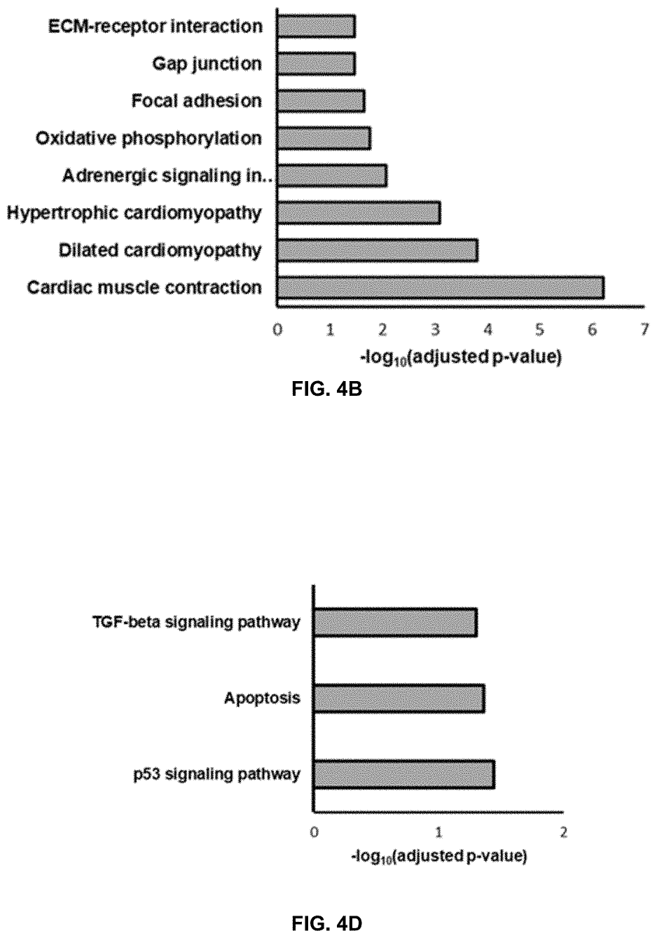

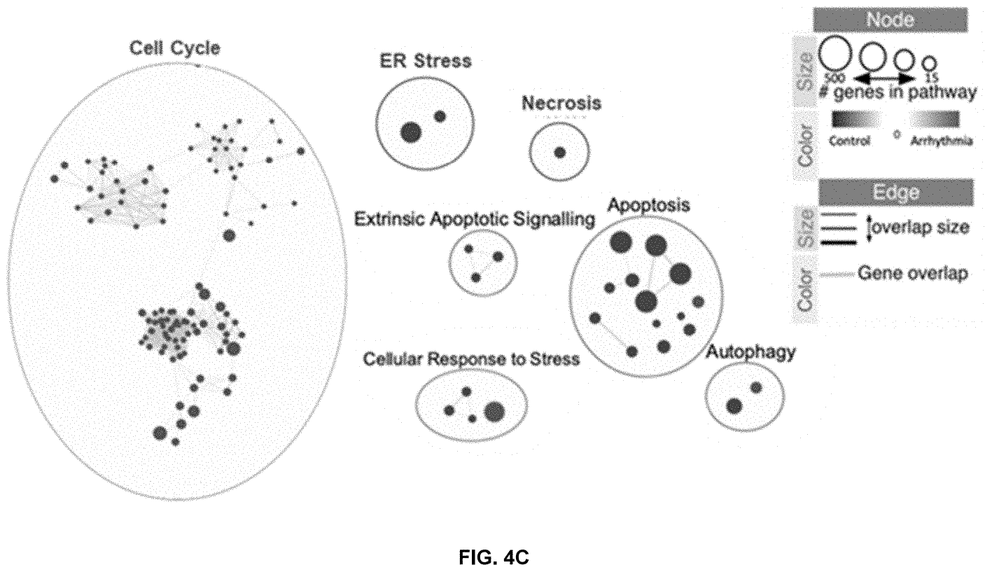

RNA was extracted from control and arrhythmic cardiac microtissues on day 5 (N=3). Gene Set Enrichment Analysis (GSEA) was performed on RPKM normalized gene expression data. GSEA showed enrichment of gene sets related to fibrosis, ion transport, cell-cell junction assembly (see FIG. 4A) in the arrhythmic samples. Conversely, gene sets related to cardiac muscle contraction and electron transport chain were enriched in the control samples (see FIG. 4A). A focused analysis of toxicity was performed (see FIG. 4B) and it was observed that arrhythmic tissues were enriched for gene sets related to Endoplasmic Reticulum (ER) stress, apoptosis, autophagy, and necrosis processes; whereas genes related to mitotic cell cycle checkpoints and nuclear division were overwhelmingly downregulated. KEGG pathway analysis was also performed separately on significantly (p-value<0.05) differentially expressed genes. Analysis demonstrated alterations in cardiac muscle contraction, oxidative phosphorylation, focal adhesion, gap junction and ECM-receptor interaction pathways (see FIG. 4B), consistent with GSEA. Interestingly, two of the top terms in this analysis were pathways involved in cardiomyopathies.

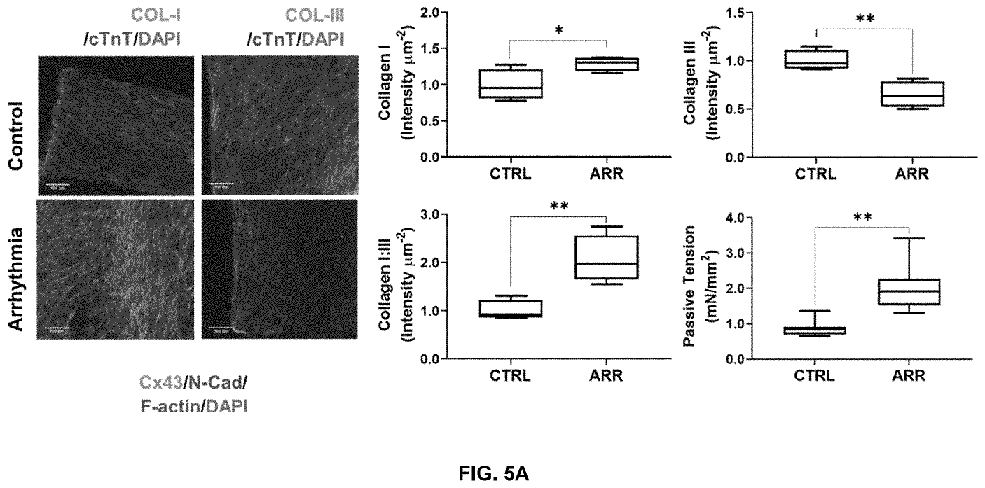

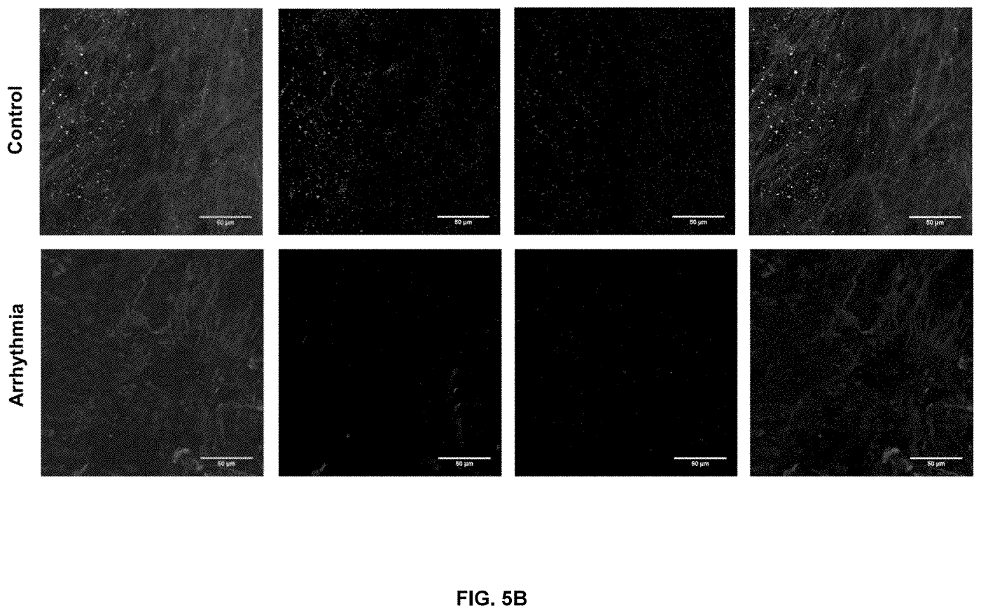

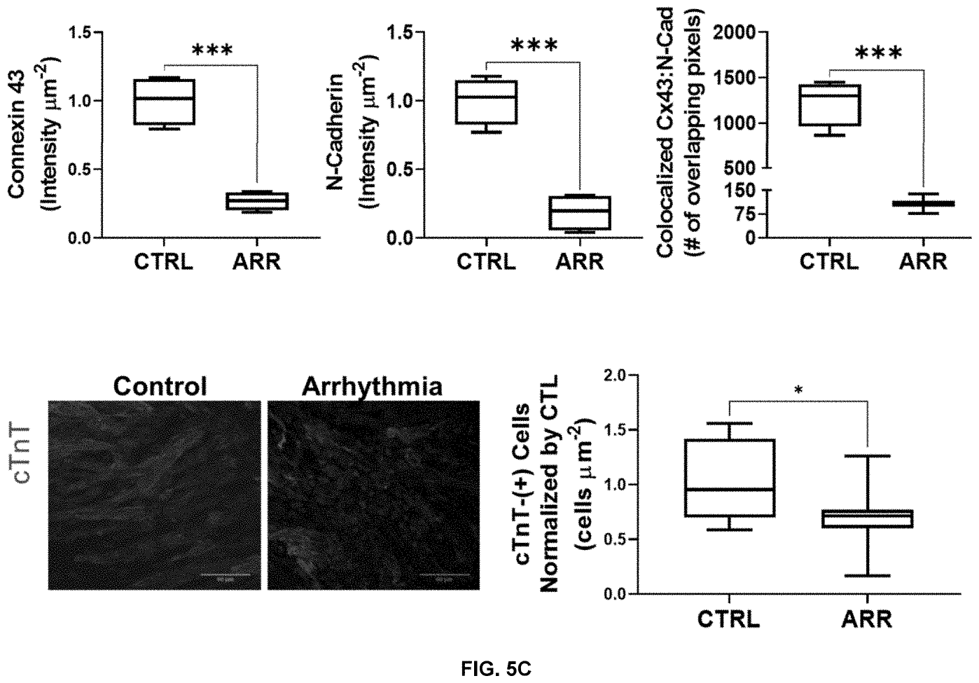

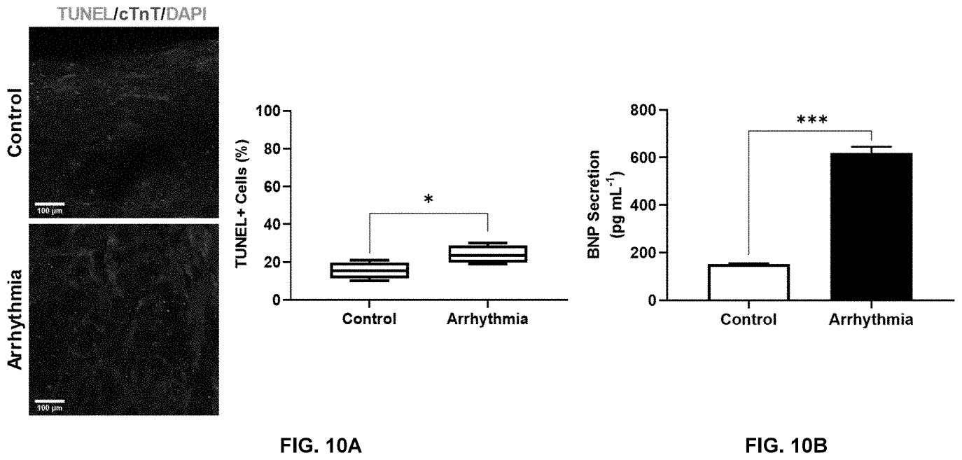

At the protein level, collagen I expression was significantly increased in arrhythmic tissues compared to control, whereas collagen III expression significantly decreased (see FIG. 5A). The ratio of collagen 1:111, as well as passive tension were significantly higher in arrhythmic tissues at day 5 compared to time matched controls (see FIG. 5A), indicating the presence of fibrosis. Furthermore, there was reduction in connexin 43, and N-cadherin expression (>50%), as well as decreased co-localization of Connexin 43 and N-cadherin (see FIG. 5B). There was also a decrease in the number of CMs in arrhythmic tissues (see FIG. 5C), in line with observed increase overall apoptosis on day 5 of treatment (see FIG. 10A). Moreover, there was a significant increase in BNP secretion from tissues treated with arrhythmogenic media at day 5 compared to controls (see FIG. 10B).

### Arrhythmia Reversal by Removal of MBCD From Media

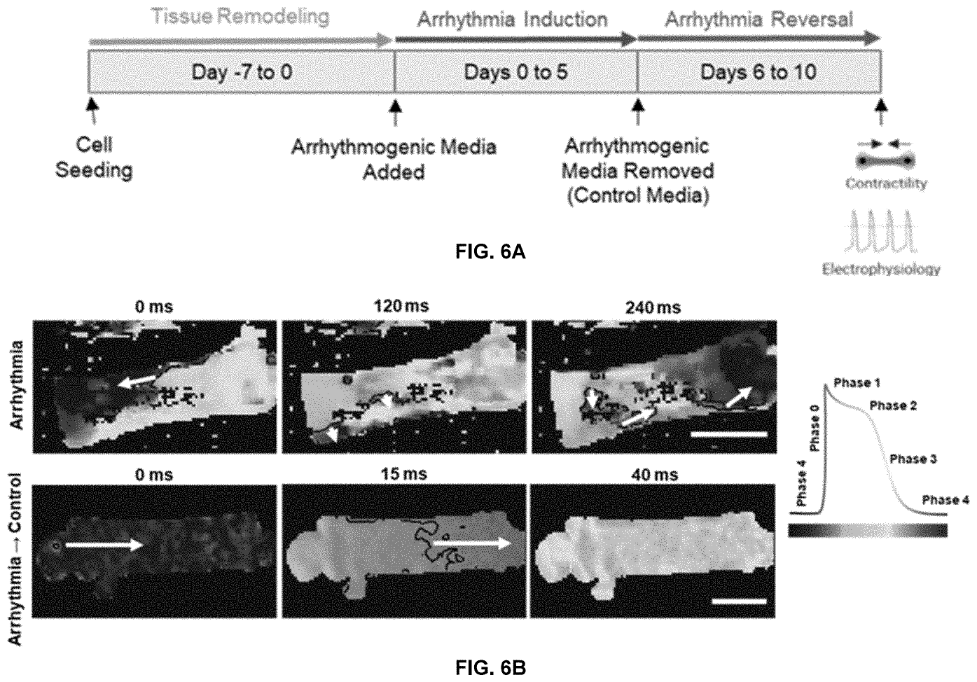

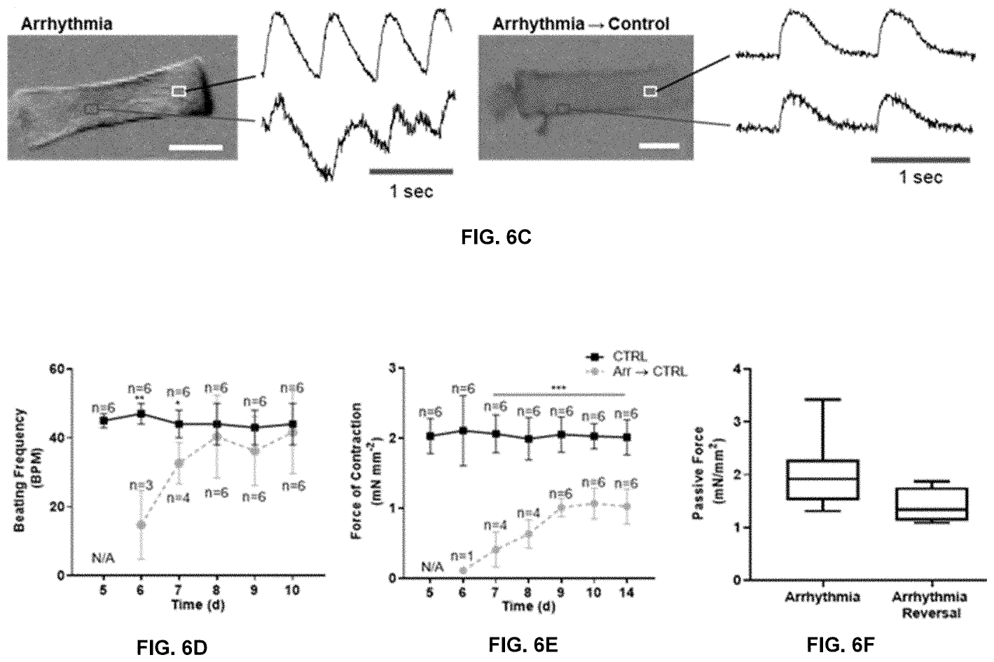

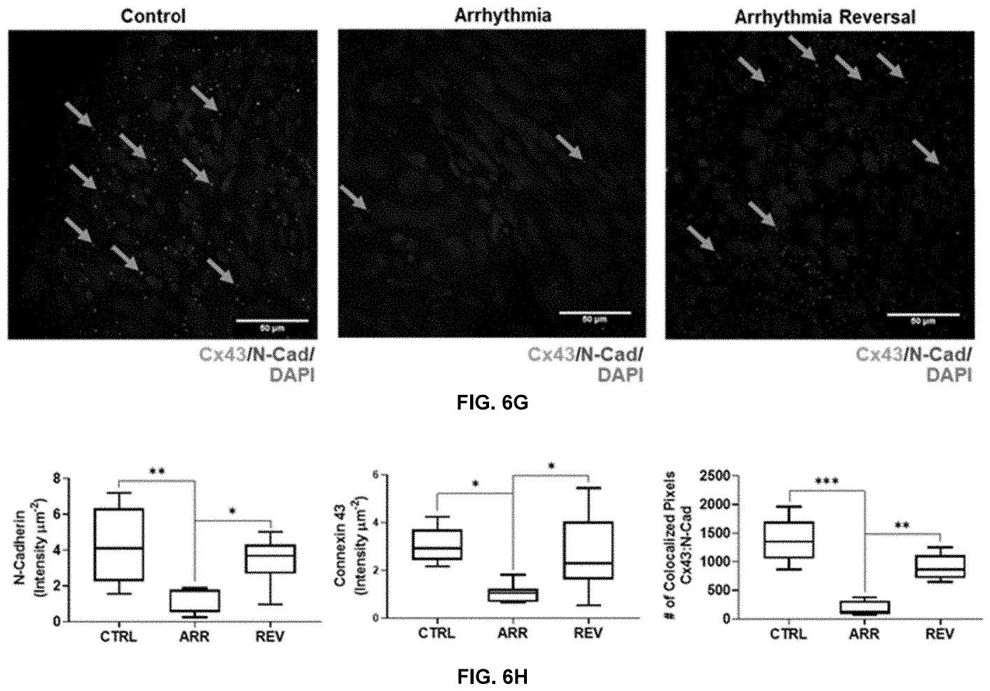

Finally, it was reasoned that if MBCD was causing arrhythmias, removal of MBCD from culture media could reverse arrhythmias. To test this, 5-day arrhythmogenic tissues were cultured in control media for 5 additional days (see FIG. 6A). Indeed, it was found that arrhythmic tissues displaying multiple wavefronts started beating synchronously, decreasing the number of mean wave fronts per frame (see FIGS. 6B-6C). Arrhythmic tissues progressively regained synchronicity, allowing for the measurement of beat frequency. Previously arrhythmic tissues treated with control media beat at a similar rate to age-matched control tissues (FIG. 6D). Assessment of the contractile force generated from the reverted tissues showed tissues did not fully regain force, plateauing at ˜50% of the contractile force generated by age matched control tissues (p<0.0001; FIG. 6D), in line with hiPSC-CM cell death reported in FIG. 4E. Arrhythmia reversal happened without significant changes in passive force (see FIG. 6E). Arrhythmia reversal was also associated with a significant increase in Cx43 and N-Cad expression and co-localization (see FIGS. 6G-6H).

To substantiate MBCD as the trigger for arrhythmias, cardiac microtissues were treated with media containing both MBCD and glucose for 5 days and the presence of arrhythmias at endpoint was assessed. It was found that, similarly to the media lacking glucose, arrhythmias were also induced in media containing both MBCD and glucose (5mM; N=3). Moreover, tissues in media containing only galactose (10 mM) but no MBCD, to rule out the possibility of arrhythmias resulting from glucose deprivation, did not become arrhythmic (N=3). It was further confirmed that MBCD was the arrhythmia inducing agent by attempting to induce arrhythmogenesis with BSA-ligated fatty acids to no effect.

### Discussion

In this study, a highly reproducible human in vitro model of acquired arrhythmia was developed using hiPSC-CMs in 3D microtissues by addition of MBCD-containing media. MBCD-triggered arrhythmias may also be induced in cardiac tissues generated with a different stem cell-derived CM line, showing the model is robust. Arrhythmogenesis was progressive, taking about 5 days to be induced in all tissues that were studied and exhibited more complex electrophysiology in later timepoints. It was found that the arrhythmic tissues of the study exhibited impaired impulse propagation with multiple wave fronts, a reduction in contractile force generation capacity, increased spontaneous beating frequency, decreased conduction velocity with a concomitant increase in action potential cycle length, decreased conduction velocity with a concomitant increase in action potential cycle length, and extensive tissue remodeling relative to controls. Notably, this model allows for the assessment of functional parameters (such as contractile force) and complex 3D electrophysiology which is not observed in other in vitro arrhythmia models [7-9] and highlights the versatility of the platform.

Recently, an elegant 3D optogenetic model of human arrhythmia by chronic tachypacing with simultaneous force measurements was described [9]. However, a few limitations remain, including the long time required to observe arrhythmias (3 weeks), the need to modify the cells to express channel rhodopsin, the need for obtaining the apparatus to perform optogenetic stimulation, the lack of sustained arrhythmias with tachycardia episodes self-terminating after 30 min from induction, and the lack of evidence for complex arrhythmias with 3D differences in wavefront migration. Moreover, arrhythmias were present in only ˜66% of the tissues, similar to other models [8,10]. However, the methodology for creating a human cardiac model described in accordance with the teachings herein rectifies these limitations by providing a robust model where all 3D tissues in the study were found to become arrhythmic in more than one hiPSC-CM line and waived the need for genetic modification of hiPSC-CMs or tachypacing, which when combined should facilitate rapid technology adoption.

The implementation of the dual electrophysiology camera system allowed the detection of 3D differential effects in wave propagation in vitro for the first time. Arrhythmias were observed to be complex in their electrophysiological nature with multiple wavefronts. Arrhythmia complexity increased with time, with increasing diversity of electrophysiological phenotypes and the increasing number of wavefronts (see FIG. 1E). This mimics the progression of arrhythmias, such as atrial fibrillation, seen in patients and in vivo models of cardiac structural and electrical remodeling due to age [26]. The observed reduction in conduction velocity and cycle length was further consistent with the pathophysiology of atrial and ventricular arrhythmias, where slower conduction velocities and shorter cycle lengths are thought to be a critical substrate for driving and maintaining the fibrillatory circuits [27, 28]. Finally, the observed higher levels of secreted BNP in arrhythmic tissues is consistent with clinical findings in severe ventricular and atrial arrhythmias [29, 30]. The sustained nature of the arrhythmias described here provided a unique opportunity to reliably test drug efficacy in arrhythmia termination.

Arrhythmic tissues responded to anti-arrhythmic drug treatment by improving calcium transient propagation (i.e., decreasing the number of wavefronts per frame). This conforms with the termination and prevention of arrhythmias such as atrial fibrillation with class I and II anti-arrhythmic drugs, where these therapies have been associated with a slowing of the conduction cycle length and conduction velocity as well as reduced automaticity in arrhythmic patients and animal models [31-34]. Furthermore, it was determined that a 2 joules biphasic pulse was sufficient to arrest the arrhythmia. This energy output is proportional to that used in human internal cardioversion (3 to 37 joules) [35].

Gene set enrichment analyses showed that there was a significant enrichment of ion transmembrane transport, cell-cell & cell-ECM junction assembly related genes in arrhythmic tissues. This was consistent with the observed decrease in intercalated disc components (Cx43/N-Cad) observed at the protein level (see FIGS. 4A and 5B) despite the low levels of connexin-43 described for hiPSC-CMs [36]. This suggests the cardiac tissue is attempting to repair itself at a transcriptomic level, which was shown to be possible via the arrhythmia reversal experiments (see FIGS. 6F and 6G). The disruption of Cx43 distribution and alignment has been shown to decrease conduction velocity and to increase the propensity of arrhythmogenesis via maladaptive cardiac conduction [37, 38]. The altered expression and/or distribution of intercalated disc proteins [28, 39], such as connexin 43 and N-cadherin, in combination with the observed disruption of cardiac electrical conduction seen in arrhythmic tissues is in line with MBCD-induced cell membrane disruption [13-15] and associated with a reduction in cell-cell communication and contractile coordination. The study also found that arrhythmic tissues exhibited a significant reduction in contractile force generation capacity (see FIG. 6D), which is consistent with the down-regulation of genes involved in electron transport chain, cardiac muscle contraction, and cardiac muscle development. Moreover, there was a significant increase in collagen I deposition, which is consistent with the observed enrichment of genes related to fibrosis and response to cytotoxicity (see FIGS. 4A-4B, 5A, 5C and 10A) reported in atrial fibrillation, heart failure, and other cardiovascular diseases associated with arrhythmias [40-42]. In sum, the observed disruption of electrical conduction ultrastructure at a transcriptomic & proteomic level is consistent with cardiac arrhythmias in vivo.

Interestingly, it was found that reintroducing arrhythmic tissues into control media reversed the arrhythmia state despite not significantly affecting fibrotic remodeling (passive tension, collagen deposition), showing a direct link between MBCD-treatment and arrhythmias. Reacquisition of synchronicity and beat rate after MBCD removal requires coordinated cellular communication and mechanical action, suggesting that arrhythmic tissues were still capable of repairing cell-cell junctions, at least to a certain degree. This was demonstrated by the increased Cx43/N-Cad expression and improved electrophysiological organization in reverted tissues in comparison to the arrhythmic state (see FIG. 6A-6H). However, the reduced contractile force in these tissues indicates that there were long-term effects. This is in agreement with the increased CM death observed in arrhythmic tissues (see FIGS. 5C and 10A) and the enrichment of cardiotoxicity and cell cycle arrest-related gene sets in arrhythmic tissues (see FIG. 4B). The lack of change in passive force in the reversed tissues suggests that fibrosis did not resolve. Together, this is consistent with the cell-cell junction/lipid raft disruption properties (see FIGS. 4A-4B) of MBCD in CMs [13, 14, 43]. Of note, despite the significant increase in passive tension and collagen deposition, characteristic of fibrosis, passive tension in arrhythmic tissues was an order of magnitude lower in the arrhythmic tissues when compared to a cardiac fibrosis model described using the same heart-on-a-chip platform and cells [44]—suggesting that the degree of fibrosis in the arrhythmic tissues is low.

In summary, a robust and reproducible 3D model of acquired human arrhythmia was developed with the use of MBCD. The model recapitulates key aspects of complex arrhythmias in vitro. Depending on the length of treatment, arrhythmias, characterized initially by tachycardia and re-entry waveforms, evolved to be more complex and clinically relevant, consisting of multiple wave fronts. Differential migration patterns were observed in 3D through the innovative use of established electrophysiology techniques and equipment. Tissues exhibiting complex arrhythmias were characterized by significantly lower conduction velocity and shorter cycle lengths, dyssynchronous twitch-like contraction, and cannot be paced by electrical stimulation—a hallmark of cardiac arrhythmias—requiring either pharmacological or electrical defibrillation for arrhythmia termination. Transcriptomic and immunohistochemistry analysis suggests that prolonged MBCD-exposure results in deleterious changes to the structural and electrical organization leading to arrhythmogenesis. Taken together, this model may be used to mimic the established pathophysiological changes seen in patients and animal models in a human-relevant platform and can serve as an investigative tool for future physiological and pharmacological research, over and above previously described models.

While the applicant's teachings described herein are in conjunction with various embodiments for illustrative purposes, it is not intended that the applicant's teachings be limited to such embodiments as the embodiments described herein are intended to be examples. On the contrary, the applicants teachings described and illustrated herein encompass various alternatives, modifications, and equivalents, without departing from the embodiments described herein, the general scope of which is defined in the appended claims.

### REFERENCES

[1] Z. Laksman, M. Wauchop, E. Lin, S. Protze, J. Lee, W. Yang, F. Izaddoustdar, S. Shafaattalab, L. Gepstein, G. F. Tibbits, G. Keller, P. H. Backx, Modeling Atrial Fibrillation using Human Embryonic Stem Cell-Derived Atrial Tissue, Sci Rep. 7 (2017). https://doi.org/10.1038/s41598-017-05652-y.
[2] J.-E. Tarride, L. Dolovich, G. Blackhouse, J. R. Guertin, N. Burke, V. Manja, A. Grinvalds, T. Lim, J. S. Healey, R. K. Sandhu, Screening for atrial fibrillation in Canadian pharmacies: an economic evaluation, Cmajo. 5 (2017) E653-E661. https://doi.org/10.9778/cmajo.20170042.
[3] M. H. Kim, S. S. Johnston, B.-C. Chu, M .R. Dalai, K. L. Schulman, Estimation of total incremental health care costs in patients with atrial fibrillation in the United States, Circ Cardiovasc Qual Outcomes. 4 (2011) 313-320. https://doi.org/10.1161/CIRCOUTCOMES.110.958165.
[4] C. Liu, A. Oikonomopoulos, N. Sayed, J. C. Wu, Modeling human diseases with induced pluripotent stem cells: from 2D to 3D and beyond, Development. 145 (2018) dev156166. https://doi.org/10.1242/dev.156166.
[5] P. Garg, V. Garg, R. Shrestha, M. C. Sanguinetti, T. J. Kamp, J. C. Wu, Human Induced Pluripotent Stem Cell-Derived Cardiomyocytes as Models for Cardiac Channelopathies: A Primer for Non-Electrophysiologists, Circ. Res. 123 (2018) 224-243. https://doi.org/10.1161/CIRCRESAHA.118.311209.
[6] M. K. Preininger, R. Jha, J. T. Maxwell, Q. Wu, M. Singh, B. Wang, A. Dalai, Z. T. Mceachin, W. Rossoll, C. M. Hales, P. S. Fischbach, M. B. Wagner, C. Xu, A human pluripotent stem cell model of catecholaminergic polymorphic ventricular tachycardia recapitulates patient-specific drug responses, Dis Model Mech. 9 (2016) 927-939. https://doi.org/10.1242/dmm.026823.
[7] M. Kawatou, H. Masumoto, H. Fukushima, G. Morinaga, R. Sakata, T. Ashihara, J. K. Yamashita, Modelling Torsade de Pointes arrhythmias in vitro in 3D human iPS cell-engineered heart tissue, Nature Communications. 8 (2017) 1078. https://doi.org/10.1038/s41467-017-01125-y.
[8] I. Goldfracht, Y. Efraim, R. Shinnawi, E. Kovalev, I. Huber, A. Gepstein, G. Arbel, N. Shaheen, M. Tiburcy, W. H. Zimmermann, M. Machluf, L. Gepstein, Engineered heart tissue models from hiPSC-derived cardiomyocytes and cardiac ECM for disease modeling and drug testing applications, Acta Biomater. 92 (2019) 145-159. https://doi.org/10.1016/j.actbio.2019.05.016.
[9] M. Lemme, I. Braren, M. Prondzynski, B. Aksehirlioglu, B. M. Ulmer, M. L. Schulze, D. Ismaili, C. Meyer, A. Hansen, T. Christ, M. D. Lemoine, T. Eschenhagen, Chronic intermittent tachypacing by an optogenetic approach induces arrhythmia vulnerability in human engineered heart tissue, Cardiovasc Res. 116 (2020) 1487-1499. https://doi.org/10.1093/cvr/cvz245.
[10] I. Goldfracht, S. Protze, A. Shiti, N. Setter, A. Gruber, N. Shaheen, Y. Nartiss, G. Keller, L. Gepstein, Generating ring-shaped engineered heart tissues from ventricular and atrial human pluripotent stem cell-derived cardiomyocytes, Nature Communications. 11 (2020) 75. https://doi.org/10.1038/s41467-019-13868-x.
[11] J. Li, L. Zhang, L. Yu, I. Minami, S. Miyagawa, M. Horning, J. Dong, J. Qiao, X. Qu, Y. Hua, N. Fujimoto, Y. Shiba, Y. Zhao, F. Tang, Y. Chen, Y. Sawa, C. Tang, L. Liu, Circulating re-entrant waves promote maturation of hiPSC-derived cardiomyocytes in self-organized tissue ring, Communications Biology. 3 (2020) 1-12. https://doi.org/10.1038/s42003-020-0853-0.
[12] K. Duval, H. Grover, L.-H. Han, Y. Mou, A. F. Pegoraro, J. Fredberg, Z. Chen, Modeling Physiological Events in 2D vs. 3D Cell Culture, Physiology (Bethesda). 32 (2017) 266-277. https://doi.org/10.1152/physio1.00036.2016.
[13] Y. Zhu, C. Zhang, B. Chen, R. Chen, A. Guo, J. Hong, L.-S. Song, Cholesterol is required for maintaining T-tubule integrity and intercellular connections at intercalated discs in cardiomyocytes, Journal of Molecular and Cellular Cardiology. 97 (2016) 204-212. https://doi.org/10.1016/j.yjmcc.2016.05.013.
[14] R. C. Balijepalli, T. J. Kamp, Caveolae, Ion Channels and Cardiac Arrhythmias, Prog Biophys Mol Biol. 98 (2008) 149-160. https://doi.org/10.1016/j.pbiomolbio.2009.01.012.
[15] C. Poulet, J. Sanchez-Alonso, P. Swiatlowska, F. Mouy, C. Lucarelli, A. Alvarez-Laviada, P. Gross, C. Terracciano, S. Houser, J. Gorelik, Junctophilin-2 tethers T-tubules and recruits functional L-type calcium channels to lipid rafts in adult cardiomyocytes, Cardiovasc Res. (2020). https://doi.org/10.1093/cvr/cvaa033.
[16] 0. Mastikhina, B.-U. Moon, K. Williams, R. Hatkar, D. Gustafson, X. Sun, M. Koo, A. Y. L. Lam, Y. Sun, J. E. Fish, E.W. K. Young, S. S. Nunes, Human cardiac-fibrosis-on-a-chip model recapitulates disease hallmarks and can serve as a platform for drug screening, BioRxiv. (2019) 632406. https://doi.org/10.1101/632406.
[17] S. S. Nunes, J. W. Miklas, J. Liu, R. Aschar-Sobbi, Y. Xiao, B. Zhang, J. Jiang, S. Masse, M. Gagliardi, A. Hsieh, N. Thavandiran, M. A. Laflamme, K. Nanthakumar, G. J. Gross, P. H. Backx, G. Keller, M. Radisic, Biowire: a platform for maturation of human pluripotent stem cell-derived cardiomyocytes, Nature Methods. 10 (2013) 781-787. https://doi.org/10.1038/nmeth.2524.
[18] S. S. Nunes, N. Feric, A. Pahnke, J. W. Miklas, M. Li, J. Coles, M. Gagliardi, G. Keller, M. Radisic, Human Stem Cell-Derived Cardiac Model of Chronic Drug Exposure, ACS Biomater. Sci. Eng. 3 (2017) 1911-1921. https://doi.org/10.1021/acsbiomaterials.5b00496.
[19] Pinto Alexander R., Ilinykh Alexei, Ivey Malina J., Kuwabara Jill T., D'Antoni Michelle L., Debuque Ryan, Chandran Anjana, Wang Lina, Arora Komal, Rosenthal Nadia A., Tallquist Michelle D., Revisiting Cardiac Cellular Composition, Circulation Research. 118 (2016) 400-409. https://doi.org/10.1161/CIRCRESAHA.115.307778.
[20] B. King, A. Porta-Sanchez, S. Masse, N. Zamiri, K. Balasundaram, M. Kusha, N. Jackson, S. Haldar, K. Umapathy, K. Nanthakumar, Effect of spatial resolution and filtering on mapping cardiac fibrillation, Heart Rhythm. 14 (2017) 608-615. https://doi.org/10.1016/j.hrthm.2017.01.023.
[21] Partek® Genomics Suite®, Partek Inc., St. Louis, 2018.
[22] U. Raudvere, L. Kolberg, I. Kuzmin, T. Arak, P. Adler, H. Peterson, J. Vilo, g:Profiler: a web server for functional enrichment analysis and conversions of gene lists (2019 update), Nucleic Acids Res. 47 (2019) W191-W198. https://doi.org/10.1093/nar/gkz369.
[23] A. Subramanian, P. Tamayo, V. K. Mootha, S. Mukherjee, B. L. Ebert, M. A. Gillette, A. Paulovich, S. L. Pomeroy, T. R. Golub, E. S. Lander, J. P. Mesirov, Gene set enrichment analysis: a knowledge-based approach for interpreting genome-wide expression profiles, Proc Natl Acad Sci U S A. 102 (2005) 15545-15550. https://doi.org/10.1073/pnas.0506580102.
[24] D. Merico, R. Isserlin, O. Stueker, A. Emili, G. D. Bader, Enrichment Map: A Network-Based Method for Gene-Set Enrichment Visualization and Interpretation, PLOS ONE. 5 (2010) e13984. https://doi.org/10.1371/journal.pone.0013984.
[25] P. Shannon, A. Markiel, O. Ozier, N. S. Baliga, J. T. Wang, D. Ramage, N. Amin, B. Schwikowski, T. Ideker, Cytoscape: a software environment for integrated models of biomolecular interaction networks, Genome Res. 13 (2003) 2498-2504. https://doi.org/10.1101/gr.1239303.
[26] D. H. Lau, L. Mackenzie, D. J. Kelly, P. J. Psaltis, A. G. Brooks, M. Worthington, A. Rajendram, D. R. Kelly, Y. Zhang, P. Kuklik, A. J. Nelson, C. X. Wong, S. G. Worthley, M. Rao, R. J. Fault, J. Edwards, D. A. Saint, P. Sanders, Hypertension and atrial fibrillation: evidence of progressive atrial remodeling with electrostructural correlate in a conscious chronically instrumented ovine model, Heart Rhythm. 7 (2010) 1282-1290. https://doi.org/10.1016/j.hrthm.2010.05.010.
[27] H.-C. Yuen, S.-Y. Roh, D.-I. Lee, J. Ahn, D.-H. Kim, J. Shim, S.-W. Park, Y.-H. Kim, Atrial fibrillation cycle length as a predictor for the extent of substrate ablation, Europace. 17 (2015) 1391-1401. https://doi.org/10.1093/europace/euu330.
[28] Igarashi Tomonori, Finet J. Emanuel, Takeuchi Ayano, Fujino Yoshihisa, Strom Maria, Greener Ian D., Rosenbaum David S., Donahue J. Kevin, Connexin Gene Transfer Preserves Conduction Velocity and Prevents Atrial Fibrillation, Circulation. 125 (2012) 216-225. https://doi.org/10.1161/CIRCULATIONAHA.111.053272.
[29] I. Sutovsky, T. Katoh, T. Ohno, H. Honma, H. Takayama, T. Takano, Relationship between brain natriuretic peptide, myocardial wall stress, and ventricular arrhythmia severity, Jpn Heart J. 45 (2004) 771-777.
[30] Hussein Ayman A., Saliba Walid I., Martin David O., Shadman Mazyar, Kanj Mohamed, Bhargava Mandeep, Dresing Thomas, Chung Mina, Callahan Thomas, Baranowski Bryan, Tchou Patrick, Lindsay Bruce D., Natale Andrea, Wazni Oussama M., Plasma B-Type Natriuretic Peptide Levels and Recurrent Arrhythmia After Successful Ablation of Lone Atrial Fibrillation, Circulation. 123 (2011) 2077-2082. https://doi.org/10.1161/CIRCULATIONAHA.110.007252.
[31] O. E. Osadchii, Effects of Na+ channel blockers on the restitution of refractory period, conduction time, and excitation wavelength in perfused guinea-pig heart, PLOS ONE. 12 (2017) e0172683. https://doi.org/10.1371/journal.pone.0172683.
[32] G. S. King, J. J. McGuigan, Antiarrhythmic Medications, in: StatPearls, StatPearls Publishing, Treasure Island (FL), 2018. http://www.ncbi.nlm.nih.gov/books/NBK482322/ (accessed Nov. 20, 2018).
[33] M. A. Allessie, M. C. Wijffels, R. Dorland, Mechanisms of pharmacologic cardioversion of atrial fibrillation by Class I drugs, J. Cardiovasc. Electrophysiol. 9 (1998) S69-77.
[34] K. C. Ravi, D. E. Krummen, A. J. Tran, J. R. Bullinga, S. M. Narayan, Electrocardiographic measurements of regional atrial fibrillation cycle length, Pacing Clin Electrophysiol. 32 Suppl 1 (2009) S66-71. https://doi.org/10.1111/j.1540-8159.2008.02229.x.
[35] J. M. del R. Sanchez, A. H. Madrid, J. M. G. Rebollo, G. P. Perez, A. Rodriguez, D. Savova, L. C. Calabria, P. Cabeza, J. D. C. Perez, M. G. Bueno, J. Mercader, E. Ripoll, C. Moro, J. M. del R. Sanchez, A. H. Madrid, J. M. G. Rebollo, G. P. Perez, A. Rodriguez, D. Savova, L. C. Calabria, P. Cabeza, J. D. C. Perez, M. G. Bueno, J. Mercader, E. Ripoll, C. Moro, External and internal electrical cardioversion: comparative, prospective evaluation of cell damage by means of troponin I, Rev Esp Cardiol. 55 (2002) 227-234.
[36] K. J. Hansen, M. A. Laflamme, G. R. Gaudette, Development of a Contractile Cardiac Fiber From Pluripotent Stem Cell Derived Cardiomyocytes, Front Cardiovasc Med. 5 (2018). https://doi.org/10.3389/fcvm.2018.00052.
[37] T. Mayama, K. Matsumura, H. Lin, K. Ogawa, I. Imanaga, Remodelling of cardiac gap junction connexin 43 and arrhythmogenesis, Exp Clin Cardiol. 12 (2007) 67-76.
[38] H. V. M. van Rijen, D. Eckardt, J. Degen, M. Theis, T. Ott, K. Willecke, H. J. Jongsma, T. Opthof, J. M. T. de Bakker, Slow conduction and enhanced anisotropy increase the propensity for ventricular tachyarrhythmias in adult mice with induced deletion of connexin43, Circulation. 109 (2004) 1048-1055. https://doi.org/10.1161/01.CIR.0000117402.70689.75.
[39] N. J. Severs, A. F. Bruce, E. Dupont, S. Rothery, Remodelling of gap junctions and connexin expression in diseased myocardium, Cardiovasc Res. 80 (2008) 9-19. https://doi.org/10.1093/cvr/cvn133.
[40] R. Ramos-Mondragón, C. A. Galindo, G. Avila, Role of TGF-beta on cardiac structural and electrical remodeling, Vasc Health Risk Manag. 4 (2008) 1289-1300.
[41] S. Sarkar, D. W. Leaman, S. Gupta, P. Sil, D. Young, A. Morehead, D. Mukherjee, N. Ratliff, Y. Sun, M. Rayborn, J. Hollyfield, S. Sen, Cardiac overexpression of myotrophin triggers myocardial hypertrophy and heart failure in transgenic mice, J. Biol. Chem. 279 (2004) 20422-20434. https://doi.org/10.1074/jbc.M308488200.
[42] G.-J. XU, T.-Y. GAN, B.-P. TANG, Z.-H. CHEN, A. MAHEMUTI, T. JIANG, J.-G. SONG, X. GUO, Y.-D. LI, H.-J. MIAO, X.-H. ZHOU, Y. ZHANG, J.-X. LI, Accelerated fibrosis and apoptosis with ageing and in atrial fibrillation: Adaptive responses with maladaptive consequences, Exp Ther Med. 5 (2013) 723-729. https://doi.org/10.3892/etm.2013.899.
[43] B. Hissa, P. W. Oakes, B. Pontes, G. Ramirez-San Juan, M. L. Gardel, Cholesterol depletion impairs contractile machinery in neonatal rat cardiomyocytes, Sci Rep. 7 (2017). https://doi.org/10.1038/srep43764.
[44] O. Mastikhina, B.-U. Moon, K. Williams, R. Hatkar, D. Gustafson, O. Mourad, X. Sun, M. Koo, A. Y. L. Lam, Y. Sun, J. E. Fish, E. W. K. Young, S. S. Nunes, Human cardiac fibrosis-on-a-chip model recapitulates disease hallmarks and can serve as a platform for drug testing, Biomaterials. 233 (2020) 119741. https://doi.org/10.1016/j.biomaterials.2019.119741.
[45] J. H. Lee, S. I. Protze, Z. Laksman, P. H. Backx, G. M. Keller, Human Pluripotent Stem Cell-Derived Atrial and Ventricular Cardiomyocytes Develop from Distinct Mesoderm Populations, Cell Stem Cell. 21 (2017) 179-194.e4. https://doi.org/10.1016/j.stem.2017.07.003.

1. A method for creating a 3D human heart model for modelling arrythmias, wherein the method comprises:
seeding a structure with a mixture of human cardiomyocytes, cardiac fibroblasts and a fibrin mixture to form cardiac tissue;
applying a plating media for settlement and compaction of the cardiac tissue; and
adding an arrhythmogenic media to the cardiac tissue, where the arrhythmogenic media comprises methyl-beta cyclodextrin for disrupting calcium signaling.

2. The method of claim 1, wherein the methyl-beta cyclodextrin is conjugated to a fatty acid.

3. The method of claim 1, wherein the arrhythmogenic media is added to the cardiac tissue about five to about nine days after the seeding is performed.

4. The method of claim 3, wherein the arrhythmogenic media is added over an increasing number of days to increase arrhythmia complexity of the cardiac tissue.

5. The method of claim 1, wherein the human cardiomyocytes comprise induced pluripotent stem cell-cardiomyocyte cells or embryonic stem cell-cardiomyocyte cells.

6. The method of claim 1, wherein the arrhythmogenic media further comprises linoleic acid, oleic acid, palmitic acid, glutamine, and/or antibiotic-antimycotic.

7. The method of claim 1, wherein the human cardiomyocytes and the cardiac fibroblasts are in a ratio from about 1:1 to about a 9:1.

8. The method of claim 1, wherein the seeding is performed during a seeding time period to prevent premature fibrin gel polymerization, where the seeding time period ranges from about 5 seconds up to about 45 seconds.

9. The method of claim 1, wherein the plating media is changed every second day.

10. The method of claim 1, wherein the plating media is applied for approximately 5 to 9 days to achieve compaction.

11. The method of claim 1, wherein the seeding comprises also using endothelial cells.

12. The method of claim 1, wherein the cardiomyocytes include nodal cells or cardiomyocytes having an atrial phenotype.

13. The method of claim 1, wherein the fibrin mixture comprises a biocompatible fibrin hydrogel.

14. The method of claim 1, wherein the structure that is seeded comprises rods in at least one microwell of a heart-on-a-chip platform.

15. A kit for performing tests on a human heart model, where the kit comprises:
a heart-on-a-chip platform comprising at least one microwell;
support elements for placement in the at least one microwell;
plating media used for settlement and compaction of the cardiac tissue; and
an arrhythmogenic media comprising methyl-beta cyclodextrin that is added during the formation of the cardiac tissue during use to disrupt calcium signaling.

16. The kit of claim 15, wherein the kit further comprises components for seeding the at least one microwell to form cardiac tissue where the components include: human cardiomyocytes, cardiac fibroblasts and a fibrin mixture;

17. The kit of claim 15, wherein the kit further comprises a fatty acid which is used to conjugate the methyl-beta cyclodextrin.

18. The kit of claim 16, wherein the components further include endothelial cells.

19. The kit of claim 16, wherein the human cardiomyocytes include nodal cells or cardiomyocytes having an atrial phenotype.

20. Use of an arrythmogenic media with a 3D human heart model for generating cardiac tissue with an arrythmia where the use comprises using methyl-beta cyclodextrin in the arrythmogenic media.
